(19) United States
(12) Patent Application Publication
(10) Pub. No.: US 2003/0008289 A1
(43) Pub. Date: Jan. 9, 2003

(54) Alleles of the human orphanin FQ/nociceptin receptor gene, diagnostic methods using said alleles, and methods of treatment based thereon

(76) Inventors: Mary Jeanne Kreek, New York, NY (US); Karl Steven LaForge, New York, NY (US)

Correspondence Address: KLAUBER & JACKSON, 411 HACKENSACK AVENUE, HACKENSACK, NJ, 07601

(21) Appl. No.: 09/905,186

(22) Filed: Oct. 9, 2001

Related U.S. Application Data
(60) Provisional application No. 60/218,205, filed on Jul. 14, 2000.

(51) Int. Cl. C12Q 1/68; C07H 21/04; C07K 14/705; C12P 21/02; C12N 5/06

(52) U.S. Cl. 435/6; 435/69.1; 435/320.1; 435/325; 530/350; 536/23.5

(57) ABSTRACT

Provided herein are variant alleles of a gene encoding a orphanin FQ/nociceptin receptor, along with cloning vectors for replicating such variant alleles, and expressing vectors for expressing the variant alleles to produce variant orphanin FQ/nociceptin receptors.

### CROSS-REFERENCE TO RELATED APPLICATION

Priority 35 U.S.C. §119(e) is claimed to U.S. provisional application serial No. 60/218,205, filed Jul. 14, 2000, incorporated herein by reference in its entirety.

### GOVERNMENTAL SUPPORT

This invention was made government support under Grant Nos. NIH-NIDA P50-DA05130, NIH-NIDA K05-DA00049, and NIH-NIDA R01-DA12848, awarded by the National Institute of Drug Addiction. The Government has certain rights in the invention.

### FIELD OF THE INVENTION

This invention relates generally to alleles of the human orphanin FQ/nociceptin receptor gene, polymorphisms thereof, methods of diagnosing various susceptibilities using such alleles and determining treatment for certain diseases based upon the presence of specific alleles, and various diseases or disorders related thereto.

### BACKGROUND OF THE INVENTION

Opioid drugs have various effects on perception of pain, consciousness, motor control, mood, autonomic function, and can also induce physical dependence. The endogenous opioid system plays an important role in modulating endocrine, cardiovascular, respiratory, gastrointestinal functions, and immune functions. Opioids, either exogenous or endogenous, exert their actions by binding to specific membrane-associated receptors.

Examples of exogenous opioids presently known include, opium, heroin, morphine, codeine, fentanyl, and methadone, to name only a few. Moreover, a family of over 20 endogenous opioid peptides has been identified, wherein the members possess common structural features, including a positive charge juxtaposed with an aromatic ring that is required for interaction with an opioid receptor. It has been determined that most, if not all the endogenous opioid peptides are derived from the proteolytic processing of three precursor proteins, i.e., pro-opiomelanocortin, proenkephalin, and prodynorphin. In addition, a fourth class of endogenous opioids, the endorphins, has been identified (the gene encoding these proteins has not yet been cloned). In the processing of the endogenous opioid precursor proteins, initial cleavages are made by membrane-bound proteases that cut next to pairs of positively charged amino acid residues, and then trimming reactions produce the final endogenous opioids secreted from cells in vivo.

Different cell types contain different processing enzymes so that, for example proopiomelanocortin can be processed into different endogenous peptides by different cells. For example, in the anterior lobe of the pituitary gland, only corticotropin (ACTH), β-lipotropin, and β-endorphin are produced. Both pro-enkephalin and pro-dynorphin are similarly processed by specific enzymes in specific cells to yield multiple opioid peptides.

Pharmacological studies have suggested there are numerous classes of opioid receptors which bind to exogenous and endogenous opioids. These classes differ in their affinity for various opioid ligands and in their cellular and organ distribution. Moreover, although the different classes are believed to serve different physiological functions, there is substantial overlap of function, as well as of distribution.

One such gene structurally related to the opioid receptor genes is the human orphanin FQ/nociceptin (also known as ORL1) receptor gene. This receptor is widely distributed in the CNS and periphery (particularly in several types of immune cells) and plays important and diverse roles in modulation of the endogenous opioid system, nociception, neurotransmitter release (including dopamine, GABA, noradrenaline, and serotonin), anxiety and stress, learning, memory and cognition, alcohol self-administration, behavioral sensitization to cocaine, drug addiction, opiate withdrawal and tolerance, food intake, immune function, cardiovascular function, renal function, gastrointestinal function, and motor function. See, for example, Bunzow J R, Saez C, Mortrud M, Bouvier C, Williams J T, Low M, Grandy D K., 1994, Molecular cloning and tissue distribution of a putative member of the rat opioid receptor gene family that is not a mu, delta or kappa opioid receptor type, FEBS Lett. 347, 284-288; Fukuda K, Kato S, Mori K, Nishi M. Takeshima H, Iwabe N, Miyata T, Houtani T Sugimoto T., 1994, cDNA cloning and regional distribution of a novel member of the opioid receptor family, FEBS Lett. 343, 42-46; Mollereau C, Parmentier M, Mailleux P, Butour J L, Moisand C, Chalon P, Caput D, Vassart G, Meunier J C., 1994, ORL1, a novel member of the opioid receptor family: Cloning functional expression and localization. FEBS Lett. 341, 33-38; Wang J B, Johnson P S, Imai Y, Persico A M, Ozenberger B A, Eppler C M, Uhl G R., 1994, cDNA cloning of an orphan opiate receptor gene family member and its splice variant FEBS Lett. 348, 75-79; Wick M J, Minnerath S R, Roy S, Ramakrishnan S, Loh H H., 1995, Expression of alternate forms of brain opioid ‘orphan’ receptor mRNA in activated human peripheral blood lymphocytes and lymphocytic cell lines, Mol Brain Res. 32, 342-347; and Peluso J, LaForge K S, Matthes H W, Kreek M J, Kieffer B L, Gavériaux-Ruff C., 1998, Distribution of nociceptin/orphanin FQ receptor transcript in human central nervous system and immune cells. J. Neuroimmunol. 81, 184-192.

The human ORL1 sequence is identified in GENBANK entries X77130, U30185, and L40949, and the wild-type nucleic acid sequence is shown in SEQ ID No: 1. The ORL1 gene was discovered based on sequence homology to the three types of opioid receptor genes (mu, delta, and kappa). The ORL1 receptor is not an opioid receptor and does not bind opioid peptides appreciably, although it exerts a modulatory effect on opioid system function, in addition to having effects on non-opioid analgesia.

It is toward the identification of alleles other than the most common or wild-type (SEQ ID No: 1) allele of the human orphanin FQ/nociceptin receptor gene, polymorphisms therein, and combinations of such polymorphisms that can be used as genetic markers to map the locus of the human orphanin FQ/nociceptin receptor gene in the genome, and additionally to correlate such polymorphisms of the human orphanin FQ/nociceptin receptor gene with susceptibility of a subject to any of the various physiological functions mentioned hereinabove in which the orphanin FQ/nociceptin receptor gene plays a role, including but not limited to determine a subject's increased or decreased susceptibility to addictive diseases, susceptibility to pain and response to analgesics, physiological responses related to the endogenous opioid system or neurotransmitter release (including dopamine, GABA, noradrenaline, and serotonin), anxiety and stress, learning, memory and cognition, alcohol self-administration, behavioral sensitization to cocaine, opiate withdrawal and tolerance, food intake, immune function, cardiovascular function, renal function, gastrointestinal function, or motor function, among other uses, that the present invention is directed.

The citation of any reference herein should not be construed as an admission that such reference is available as “Prior Art” to the instant application.

### SUMMARY OF THE INVENTION

There is provided, in accordance with the present invention, heretofore unknown single-nucleotide polymorphisms (SNPs) of the human orphanin FQ/nociceptin receptor gene, and their use in mapping the locus of the human orphanin FQ/nociceptin receptor gene; determining susceptibility to addictive diseases; determining susceptibility to pain; determining a therapeutically effective amount of pain reliever to administer to a subject suffering from pain; diagnosing a disease or disorder in a subject related to a physiological response, condition or disorder such as but not limited to nociception, neurotransmitter release (including dopamine, GABA, noradrenaline, and serotonin), anxiety and stress, learning, memory and cognition, alcohol self-administration, behavioral sensitization to cocaine, drug addiction, opiate withdrawal and tolerance, food intake, immune function, cardiovascular function, renal function, gastrointestinal function, and motor function; and selecting an appropriate therapeutic agent and a therapeutically effective amount of such an agent to administer to a subject suffering from an aforementioned disease or disorder. One or more of the polymorphisms of the invention may be employed as such; and an individual may have one or more of the polymorphisms. Moreover, the polymorphisms individually and in combination may be present homozygously or heterozygously.

The polymorphisms of the human orphanin FQ/nociceptin receptor gene described herein are G-46A (G minus 46 A), located in the 5′ untranslated region; GIVS I 135C, located in intron I; GIVS I 250A, located in intron I; GIVS I 251A, located in intron I; C510T, a silent mutation located in the coding region; CIVS III 67T; located in intron III; A804G, a silent mutation located in the coding region; C1026T, a silent mutation located in the coding region; and C1126G, located in the 3′ untranslated region.

The present invention extends to DNA sequences of heretofore unknown isolated nucleic acid molecules which encode human orphanin FQ/nociceptin receptors, wherein the DNA sequences include any combination of the aforementioned known polymorphisms.

The present invention further extends to diagnostic methods to determine a subject's increased or decreased susceptibility to the aforementioned conditions, diseases, and physiological responses. With the results of such methods, targeted prevention methods, early therapeutic intervention, and improved chronic treatment are set forth herein and encompassed by the present invention. In addition, attending medical professionals armed with the results of such diagnostic methods can determine, for example, whether administration of opioid analgesics is appropriate or whether non-opioid derived analgesics should be administered to the subject. Furthermore, appropriate choice and type of analgesic to treat a subject's pain can be made. Such determination may be made by identification of any individual or any combination of the above-mentioned polymorphisms, using such non-limiting methods as DNA sequencing, differential hybridization to biological chip arrays such as an oligonucleotide gelpad microchip, or single nucleotide extension (SNE) on chip arrays such as on oligonucleotide gelpad microchips.

Also, the present invention extends to methods of determining a subject's increased or decreased susceptibility to pain and response to analgesics, and the use of the information in prescribing analgesics to the subject.

Broadly the present invention extends to an isolated variant allele of a human orphanin FQ/nociceptin receptor gene which can serve as a genetic marker, wherein the predominant or “most common” allele of a human orphanin FQ/nociceptin receptor gene found in the population comprises a DNA sequence of SEQ ID NO:1, and a variant allele of the present invention comprises a DNA sequence having a variation in SEQ ID NO:1, wherein the variation comprises G-46A, GIVS I 135C, GIVS I 250A, GIVS I 251A, C510T, CIVS III 67T, A804G, C1026T, or C1126G, or any combination thereof.

Furthermore, the present invention extends to an isolated variant allele of a human orphanin FQ/nociceptin receptor gene as set forth above, which is detectably labeled. Numerous detectable labels have applications in the present invention, such as radioactive elements, chemicals which fluoresces, or enzymes, to name only a few.

The present invention further extends to an isolated nucleic acid molecule selectively hybridizable to an isolated variant allele of the human orphanin FQ/nociceptin receptor gene, wherein the predominant or “most common” allele of a human orphanin FQ/nociceptin receptor gene found in the population comprises a DNA sequence of SEQ ID NO:1, and a variant allele of the present invention comprises a DNA sequence having a variation in SEQ ID NO:1, wherein the variation comprises G-46A, GIVS I 135C, GIVS I 250A, GIVS I 251A, C510T, CIVS III 67T, A804G, C1026T, or C1126G, or any combination thereof.

Moreover, the present invention extends to an isolated nucleic acid molecule selectively hybridizable to an isolated variant allele of the human orphanin FQ/nociceptin receptor gene, wherein the predominant or “most common” allele of a human orphanin FQ/nociceptin receptor gene found in the population comprises a DNA sequence of SEQ ID NO:1, and a variant allele of the present invention comprises a DNA sequence having a variation in SEQ ID NO:1, wherein the variation comprises G-46A, GIVS I 135C, GIVS I 250A, GIVS I 251A, C510 T, CIVS III 67T, A804G, C1026T, or C1126G, or any combination thereof, wherein the isolated nucleic acid molecule is detectably labeled. Examples of detectable labels that have applications in this embodiment of the present invention are described above.

In addition, the present invention extends to cloning vectors that can be used to clone copies of a variant alleles of a human orphanin FQ/nociceptin receptor gene of the present invention. For example, the present invention extends to a cloning vector comprising an isolated variant allele of a human orphanin FQ/nociceptin receptor gene and an origin of replication, wherein the predominant or “most common” allele of a human orphanin FQ/nociceptin receptor gene found in the population comprises a DNA sequence of SEQ ID NO:1, and a variant allele of the present invention comprises a DNA sequence having a variation in SEQ ID NO:1, wherein the variation comprises G-46A, GIVS I 135C, GIVS I 250A, GIVS I 251A, C510T, CIVS III 67T, A804G, C1026T, or C1126G, or any combination thereof.

In another embodiment, the present invention extends to a cloning vector comprising an isolated nucleic acid molecule selectively hybridizable to an isolated variant allele of a human orphanin FQ/nociceptin receptor gene, and an origin of replication, wherein the predominant or “most common” allele of a human orphanin FQ/nociceptin receptor gene found in the population comprises a DNA sequence of SEQ ID NO:1, and a variant allele of the present invention comprises a DNA sequence having a variation in SEQ ID NO:1, wherein the variation comprises G-46A, GIVS I 135C, GIVS I 250A, GIVS I 251A, C510T, CIVS III 67T, A804G, C1026T, or C1126G, or any combination thereof.

Numerous cloning vectors have applications in the present invention. For example, a cloning vector having applications in the present invention includes E. coli, bacteriophages such as lambda derivatives, plasmids such as pBR322 derivatives, and pUC plasmid derivatives such as pGEX vectors or pmal-c or pFLAG, to name only a few.

Naturally, the present invention extends to expression vectors comprising an isolated variant allele a human orphanin FQ/nociceptin receptor gene operatively associated with a promoter, wherein the predominant or “most common” allele of a human orphanin FQ/nociceptin receptor gene found in the population comprises a DNA sequence of SEQ ID NO:1, and a variant allele of the present invention comprises a DNA sequence having a variation in SEQ ID NO:1, wherein the variation comprises: G-46A, GIVS I 135C, GIVS I 250A, GIVS I 251A, C510T, CIVS III 67T, A804G, C1026T, or C1126G, or any combination thereof.

Furthermore, the present invention extends to an expression vector comprising an isolated nucleic acid molecule selectively hybridizable to an isolated variant allele a human orphanin FQ/nociceptin receptor gene, wherein the isolated nucleic acid molecule is operatively associated with a promoter. As set forth above, the predominant or “most common” allele of a human orphanin FQ/nociceptin receptor gene found in the population comprises a DNA sequence of SEQ ID NO:1, and a variant allele of the present invention comprises a DNA sequence having a variation in SEQ ID NO:1, wherein the variation comprises G-46A, GIVS I 135C, GIVS I 250A, GIVS I 251A, C510T, CIVS III 67T, A804G, C1026T, or C1126G, or any combination thereof.

Numerous promoters have applications in an expression vector of the present invention, including but not limited to immediate early promoters of hCMV, early promoters of SV40, early promoters of adenovirus, early promoters of vaccinia, early promoters of polyoma, late promoters of SV40, late promoters of adenovirus, late promoters of vaccinia, late promoters of polyoma, the lac the trp system, the TAC system, the TRC system, the major operator and promoter regions of phage lambda, control regions of fd coat protein, 3-phosphoglycerate kinase promoter, acid phosphatase promoter, or promoters of yeast α mating factor, to name only a few.

In addition, the present invention extends to a unicellular host transformed or transfected with an expression vector of the present invention. Examples of hosts which can be transformed or transfected with an expression vector of the present invention, and have applications in the present invention, include, but are not limited to, E. coli, Pseudomonas, Bacillus, Streptomyces, yeast, CHO, R1.1, B-W, L-M, COS1, COS7, BSC1, BSC40, BMT10 or Sf9 cells.

The invention further extends to altered expression of the wild-type orphanin FQ/nociceptin gene product, and means for detecting the altered expression, as a consequence of the presence of any one or any combination of the polymorphisms G-46A, GIVS I 135C, GIVS I 250A, GIVS I 251A, C510T, CIVS III 67T, A804G, C1026T, or C1126G.

Accordingly, the present invention extends to a method for determining a susceptibility in a subject to at least one disease, comprising the steps of removing a bodily sample comprising a first and second allele of a human orphanin FQ/nociceptin receptor gene from the subject, and determining whether the first allele comprises a human orphanin FQ/nociceptin receptor gene comprising a DNA sequence having at least one variation in SEQ ID NO:1, wherein the variation comprises G-46A, GIVS I 135C, GIVS I 250A, GIVS I 251A, C510T, CIVS III 67T, A804G, C1026T, or C1126G.

The present of at least one of these variations in the human orphanin FQ/nociceptin receptor gene of the first allele is expected to be indicative of the subject's susceptibility to at least one disease relative to the susceptibility of a standard, wherein the standard comprises a first allele comprising a human orphanin FQ/nociceptin receptor gene having a DNA sequence of SEQ ID NO:1.

Another embodiment of the method for determining a susceptibility in the subject to at least one disease, as described above, comprises the further step of determining whether the second allele of the bodily sample of the subject comprises a human orphanin FQ/nociceptin receptor gene comprising a DNA sequence having at least one variation in SEQ ID NO:1, wherein the variations comprise G-46A, GIVS I 135C, GIVS I 250A, GIVS I 251A, C510T, CIVS III 67T, A804G, C1026T, or C1126G.

Furthermore, the present invention extends to a method for determining a susceptibility to pain in a subject relative to susceptibility to pain in a standard, comprising the steps of removing a bodily sample comprising a first and second allele of a human orphanin FQ/nociceptin receptor gene from the subject, and determining whether the first allele comprises a human orphanin FQ/nociceptin receptor gene comprising a DNA sequence having at least one variation in SEQ ID NO:1, wherein the variation comprises one or more of the polymorphisms G-46A, GIVS I 135C, GIVS I 250A, GIVS I 251A, C510T, CIVS III 67T, A804G, C1026T, or C1126G. The presence of at least one variation in the human orphanin FQ/nociceptin receptor gene of the first allele is expected to be indicative of a decreased or increased susceptibility to pain in the subject relative to susceptibility to pain in the standard, wherein the first allele of the standard comprises a human orphanin FQ/nociceptin receptor gene comprising a DNA sequence of SEQ ID NO:1.

Moreover, a method for determining a susceptibility to pain in a subject may further comprise the step of determining whether the second allele comprises a human orphanin FQ/nociceptin receptor gene comprising a DNA sequence having at least one variation in SEQ ID NO:1, wherein the variation comprises one or more of the polymorphisms G-46A, GIVS I 135C, GIVS I 250A, GIVS I 251A, C510T, CIVS III 67T, A804G, C1026T, or C1126G. The presence of the at least one variation in the human orphanin FQ/nociceptin receptor gene of the second allele of the bodily sample from the subject is expected to be indicative of an increased or decreased susceptibility to pain in the subject relative to the susceptibility to pain in the standard, wherein the second allele in the standard comprises a human orphanin FQ/nociceptin receptor gene comprising a DNA sequence of SEQ ID NO:1.

Consequently, the present invention extends to a method for determining a therapeutically effective amount of pain reliever to administer to a subject in order to induce analgesia in the subject relative to a therapeutically effective amount of the pain reliever to administer to a standard in order to induce analgesia in the standard, wherein the method comprises determining a susceptibility to pain in the subject relative to susceptibility to pain in the standard. The susceptibility of pain in the subject is expected to be indicative of the therapeutically effective amount of the pain reliever to administer to the subject to induce analgesia in the subject relative to the amount of the pain reliever to administer to the standard to induce analgesia in the standard.

Hence, the present invention extends to a method for determining a therapeutically effective amount of pain reliever to administer to a subject in order to induce analgesia in the subject relative to a therapeutically effective amount of the pain reliever to administer to a standard in order to induce analgesia in the standard wherein the method comprises the steps of removing a bodily sample comprising a first and second allele of a human orphanin FQ/nociceptin receptor gene from the subject, and determining whether the first allele comprises a human orphanin FQ/nociceptin receptor gene comprising a DNA sequence having at least one variation in SEQ ID NO:1, wherein the at least one variation comprises G-46A, GIVS I 135C, GIVS I 250A, GIVS I 251A, C510T, CIVS III 67T, A804G, C1026T, or C1126G. The presence of at least one variation in the human orphanin FQ/nociceptin receptor gene of the first allele from the bodily sample is expected to be indicative of the therapeutically effective amount of pain reliever to administer to the subject to induce analgesia in the subject relative to the therapeutically effective amount of pain reliever to administer to the standard to induce analgesia in the standard, wherein the standard comprises a first allele comprising a human orphanin FQ/nociceptin receptor gene comprising a DNA sequence of SEQ ID NO:1.

Moreover, the present invention further extends to a method for determining a therapeutically effective amount of pain reliever to administer to a subject in order to induce analgesia in the subject relative to a therapeutically effective amount of pain reliever to administer to a standard to induce analgesia therein, further comprising the steps of removing a bodily sample comprising a first and second allele comprising a human orphanin FQ/nociceptin receptor gene from the subject, and determining whether the second allele of the bodily sample comprises a human orphanin FQ/nociceptin receptor gene comprising a DNA sequence comprising at least one variation in SEQ ID NO:1, wherein the at least one variation comprises G-46A, GIVS I 135C, GIVS I 250A, GIVS I 251A, C510T, CIVS III 67T, A804G, C1026T, or C1126G. The presence of at least one variation in the human orphanin FQ/nociceptin receptor gene of the first and/or second allele of the bodily sample is expected to be indicative of the therapeutically effective amount of pain reliever to administer to the subject to induce analgesia therein relative to the amount of pain reliever to administer to a standard to induce analgesia therein, wherein the first and second alleles of the standard comprise a human orphanin FQ/nociceptin receptor gene comprising a DNA sequence of SEQ ID NO:1.

Examples of pain relievers having applications in this embodiment of the present invention include, but are not limited to, morphine, codeine, dihydromorphin, meperidine, methadone, fentanyl and its congeners, butorphenol, nalbuphine, LAAM, or propoxyphine, to name only a few.

The present invention further extends to commercial test kits suitable for use by a medical professional to determine whether either or both alleles of a bodily sample taken from a subject comprise a DNA sequence having at least one variation in SEQ ID NO:1, wherein the variation 2 comprises G-46A, GIVS I 135C, GIVS I 250A, GIVS I 251A, C510T, CIVS III 67T, A804G, C1026T, or C1126G.

Commercial test kits of the present invention have applications in determining susceptibility of pain in the subject relative to a standard. Such kits can also be used to determine a subject's increased or decreased susceptibility to at least one addictive disease relative to susceptibility to at least one addictive disease in a standard. Also a therapeutically effective amount of pain reliever to administer to the subject in order to induce analgesia in the subject relative to a therapeutically effective amount of pain reliever to administer to a standard to induce analgesia in the standard can be determined. Moreover, a test kit of the present invention has applications in determining a therapeutically effective amount of therapeutic agent for treating at least one physiological response, condition or disease to administer to a subject suffering therefrom, relative to a therapeutically effective amount of therapeutic agent to administer to a standard.

Furthermore, a commercial test kit of the present invention can also be used to determine the presence of an isolated variant allele of a human orphanin FQ/nociceptin receptor gene of the present invention in a bodily sample removed from a subject, which can serve as a genetic marker. As explained above, the predominant or “most common” allele of a human orphanin FQ/nociceptin receptor gene found in the population comprises a DNA sequence of SEQ ID NO:1. Hence a variant allele comprising a DNA sequence having a variation in SEQ ID NO:1, wherein the variation comprises G-46A, GIVS I 135C, GIVS I 250A, GIVS I 251A, C510T, CIVS III 67T, A804G, C1026T, C1126G, or combinations thereof, can be detected in the bodily sample with a commercial kit of the invention.

Accordingly, a commercial test kit may be prepared for determining the presence of at least one variation in a human orphanin FQ/nociceptin receptor gene of either or both alleles in a bodily sample taken from a subject, wherein the commercial test kit comprises:

a) PCR oligonucleotide primers suitable for detection of an allele comprising a human orphanin FQ/nociceptin receptor gene having a DNA sequence with a variation in SEQ ID NO:1;

b) other reagents; and

c) directions for use of the kit.

Accordingly, the present invention extends to a commercial test kit having applications set forth above, comprising a predetermined amount of at least one detectably labeled immunochemically reactive component having affinity for a variant human orphanin FQ/nociceptin receptor;

(b) other reagents; and

(c) directions for use of the kit.

In a further variation, the test kit may be prepared and used for the purposes stated above, which operates according to a predetermined protocol (e.g. “competitive,” “sandwich,” “double antibody,” etc.), and comprises:

(a) a labeled component which has been obtained by coupling the human orphanin FQ/nociceptin receptor of a bodily sample to a detectable label;

(b) one or more additional immunochemical reagents of which at least one reagent is a ligand or an immobilized ligand, which ligand comprises:

(i) a ligand capable of binding with the labeled component (a);

(ii) a ligand capable of binding with a binding partner of the labeled component (a);

(iii) a ligand capable of binding with at least one of the component(s) to be determined; or

(iv) a ligand capable of binding with at least one of the binding partners of at least one of the component(s) to be determined; or

(c) directions for the performance of a protocol for the detection and/or determination of one or more components of an immunochemical reaction between the human orphanin FQ/nociceptin receptor gene of the present invention and a specific binding partner thereto.

The present invention is also directed to the finding of a novel 511-nucleotide intron between bases -34 and -33 of the orphanin FQ/nociceptin receptor gene mRNA, herein designated “Intervening Sequence I (IVS I)” (SEQ ID No:2).

Accordingly, it is an object of the present invention to provide heretofore unknown variations the DNA sequence of the human orphanin FQ/nociceptin receptor gene wherein the variations can be used to map the locus of the human orphanin FQ/nociceptin receptor gene.

It is yet another object of the present invention to use heretofore unknown polymorphisms of an allele of the human orphanin FQ/nociceptin receptor gene as markers for any kind of disorder related to the human orphanin FQ/nociceptin receptor, such as an addictive disease, pain, or markers for genes.

It is another object of the present invention to provide nucleotides, optionally detectably labeled, selectively hybridizable to variant alleles of the human orphanin FQ/nociceptin receptor gene disclosed herein, as well as polypeptides produced from the expression of the variant alleles and nucleotides selectively hybridizable thereto under selective hybridization conditions.

It is another object of the present invention to gain insight into a subject's susceptibility to pain. This insight can be used to determine a therapeutically effective dose of pain reliever to administer to the subject to induce analgesia therein relative to the therapeutically effective amount of pain reliever administered to a standard to induce analgesia therein, wherein the standard comprises two alleles of the human orphanin FQ/nociceptin receptor gene comprising a DNA sequence of SEQ ID NO:1.

Such information can be used to tailor a regimen for treating a subject suffering from at least one addictive disease, relative to the therapeutically effective amount of therapeutic agent administered to a standard suffering from at least one addictive disease.

It is yet another object of the present invention to provide commercial test kits for attending medical professionals to determine the presence of variant alleles of a human orphanin FQ/nociceptin receptor gene in a bodily sample taken from a subject. The results of such testing can then be used to determine the subject's nociception, neurotransmitter release (including dopamine, GABA, noradrenaline, and serotonin), anxiety and stress, learning, memory and cognition, alcohol self-administration, behavioral sensitization to cocaine, drug addiction, opiate withdrawal and tolerance, food intake, immune function, cardiovascular function, renal function, gastrointestinal function, and motor function, determining a therapeutically effective amount of pain reliever to administer to the subject in order to induce analgesia, or determining a therapeutically effective amount of therapeutic agent for treating at least one addictive disease to administer to the subject.

It is yet another object of the present invention to provide commercial detecting variant alleles of the human orphanin FQ/nociceptin receptor gene or the presence of a variant human orphanin FQ/nociceptin receptor in a bodily sample taken from a subject. The results of such tests can then be used to gain incite into a subject's ability to withstand pain, susceptibility to addiction, to diagnose a disease or disorder related to nociception, neurotransmitter release (including dopamine, GABA, noradrenaline, and serotonin), anxiety and stress, learning, memory and cognition, alcohol self-administration, behavioral sensitization to cocaine, drug addiction, opiate withdrawal and tolerance, food intake, immune function, cardiovascular function, renal function, gastrointestinal function, and motor function.

These and other aspects of the present invention will be better appreciated by reference to the following drawings and Detailed Description.

### BRIEF DESCRIPTION OF THE DRAWINGS

FIG. 1 depicts the nucleic acid sequence of the most common allele of the human orphanin FQ/nociceptin receptor gene (SEQ ID NO:1) (GENBANK accession number X77130, U30185 or L40949).

FIG. 2 depicts the nucleic acid sequence of the 511-nucleotide intron herein designated Intervening Sequence I (IVS I; intron I) located between bases -34 and -33 of the human orphanin FQ/nociceptin receptor mRNA (SEQ ID No:2).

FIG. 3 depicts the nucleic acid sequence of the G-46A polymorphism in the 5′-untranslated region of the human orphanin FQ/nociceptin receptor gene (SEQ ID NO:3).

FIG. 4 depicts the nucleic acid sequence of the GIVS I 135C polymorphism in intron I of the human orphanin FQ/nociceptin receptor (SEQ ID NO:4).

FIG. 5 depicts the nucleic acid sequence of the GIVS I 250A polymorphism in intron I of the human orphanin FQ/nociceptin receptor gene (SEQ ID NO:5).

FIG. 6 depicts the nucleic acid sequence of the GIVS I 251 A polymorphism in intron I of the human orphanin FQ/nociceptin receptor gene (SEQ ID NO:6).

FIG. 7 depicts the nucleic acid sequence of the C510T polymorphism in the coding region of the human orphanin FQ/nociceptin receptor gene (SEQ ID NO:7).

FIG. 8 depicts the nucleic acid sequence of the CIVS III 67T polymorphism in intron III of the human orphanin FQ/nociceptin receptor gene (SEQ ID NO:8).

FIG. 9 depicts the nucleic acid sequence of the A804G polymorphism in the coding region of the human orphanin FQ/nociceptin receptor gene (SEQ ID NO:9).

FIG. 10 depicts the nucleic acid sequence of the C 1026T polymorphism in the coding region of the human orphanin FQ/nociceptin receptor gene (SEQ ID NO:10).

FIG. 11 depicts the nucleic acid sequence of the C1126G polymorphism in the 3′-untranslated region of the human orphanin FQ/nociceptin receptor gene (SEQ ID NO:11).

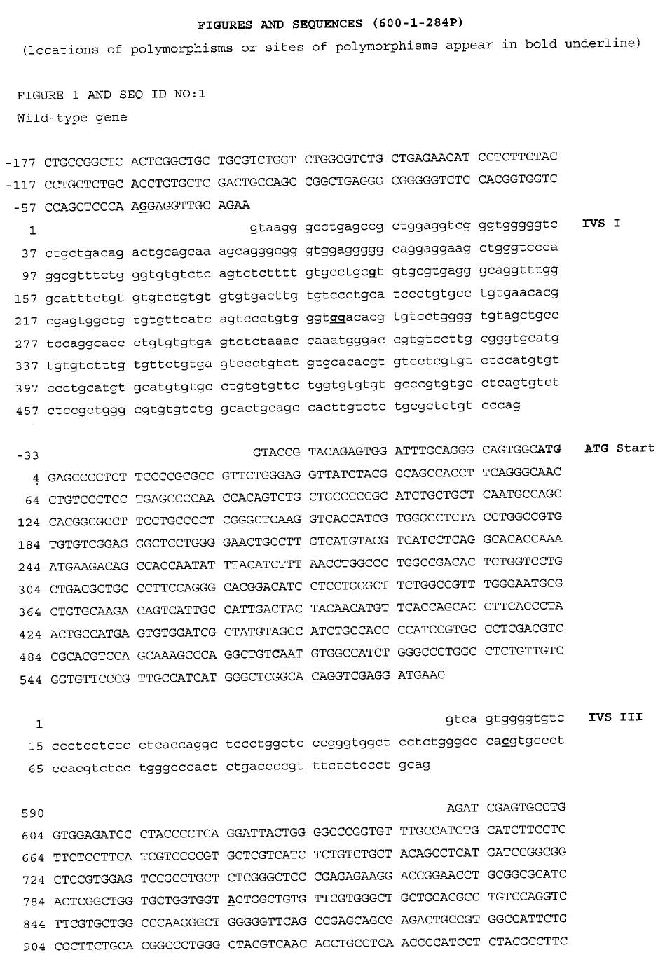

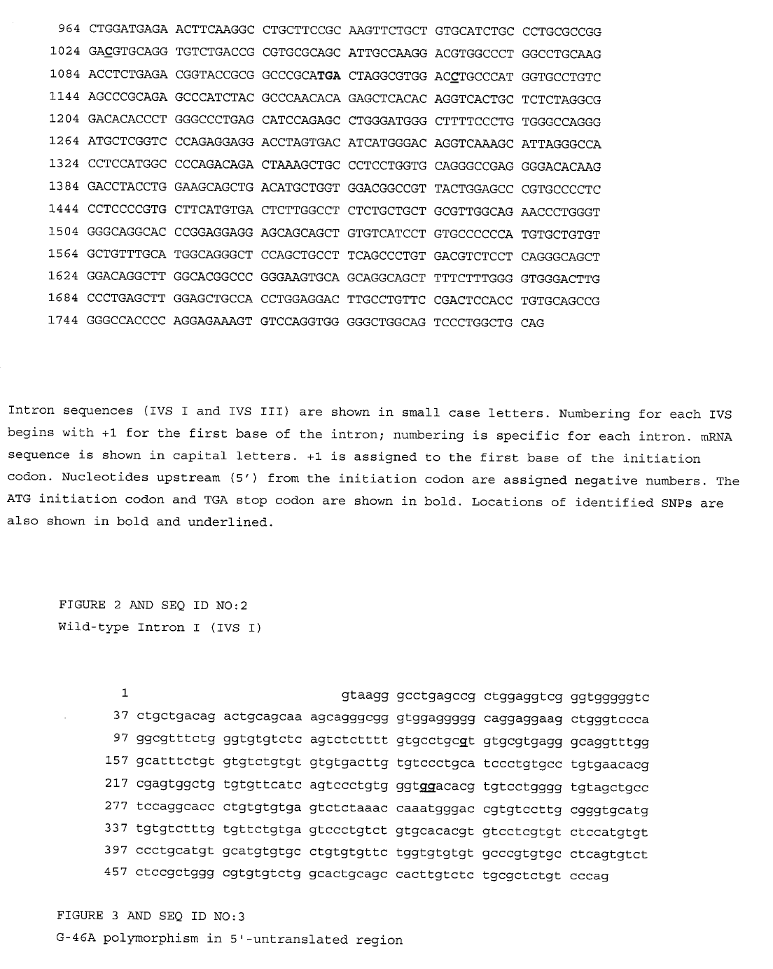

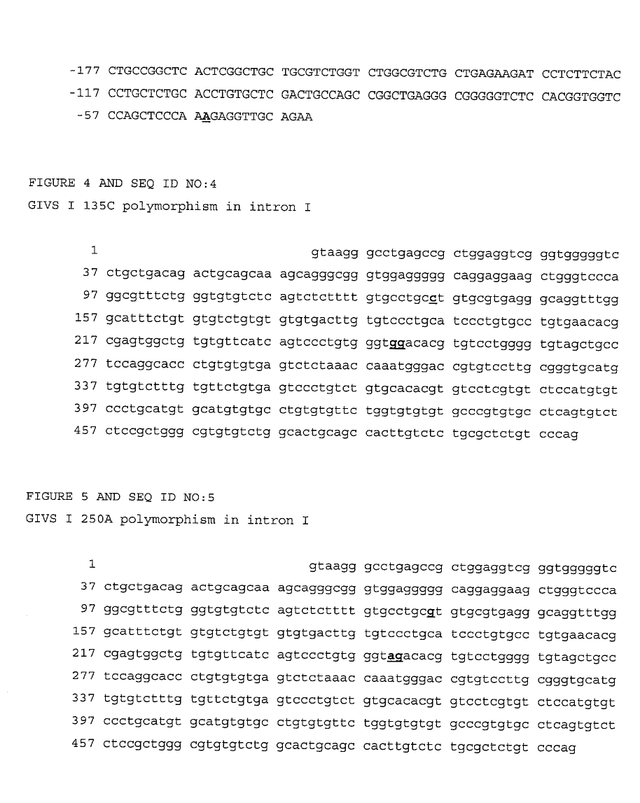

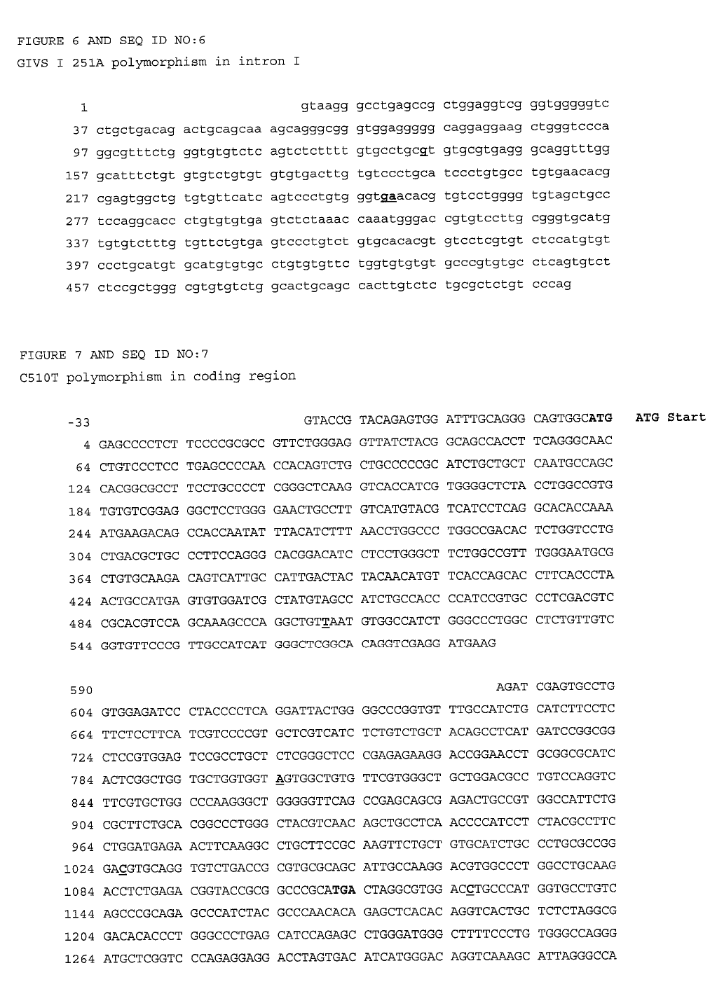

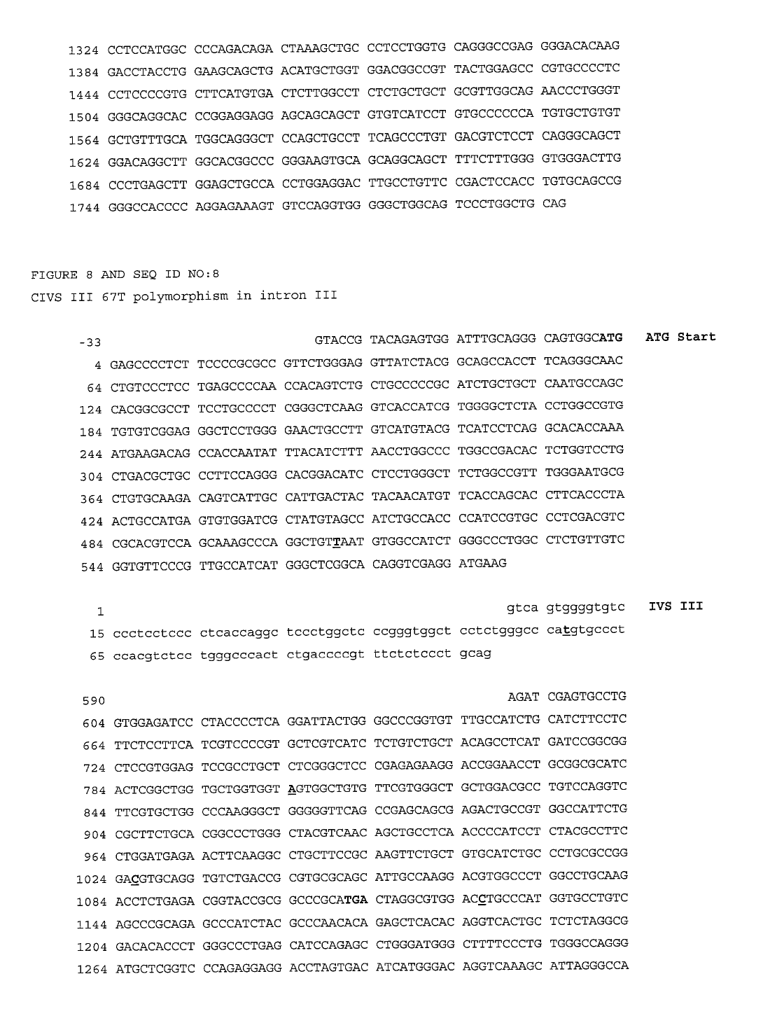

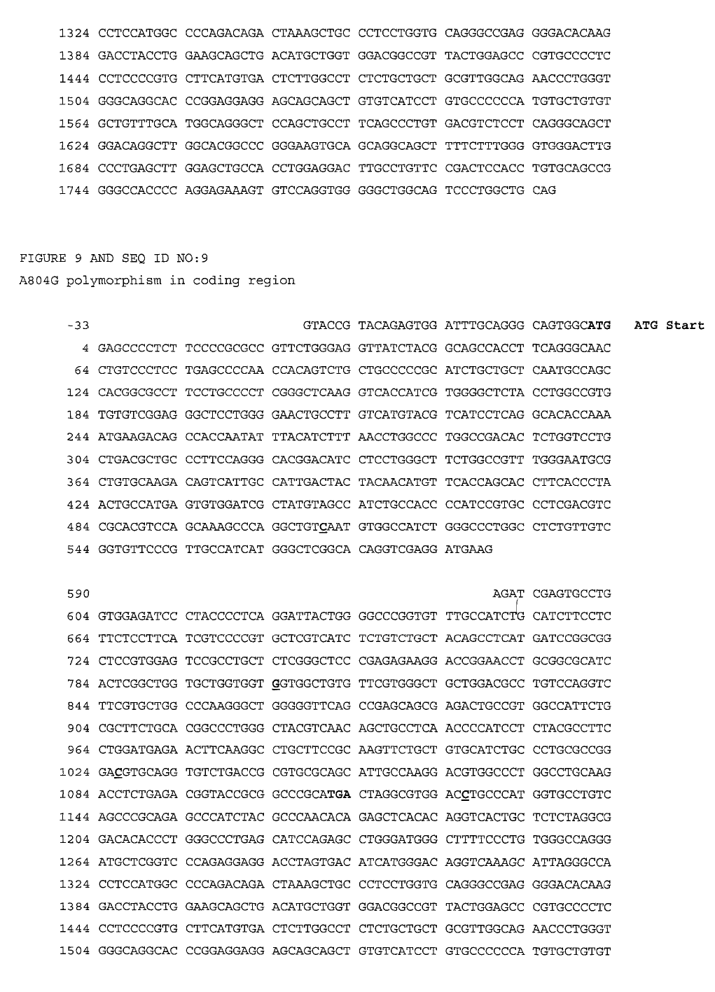

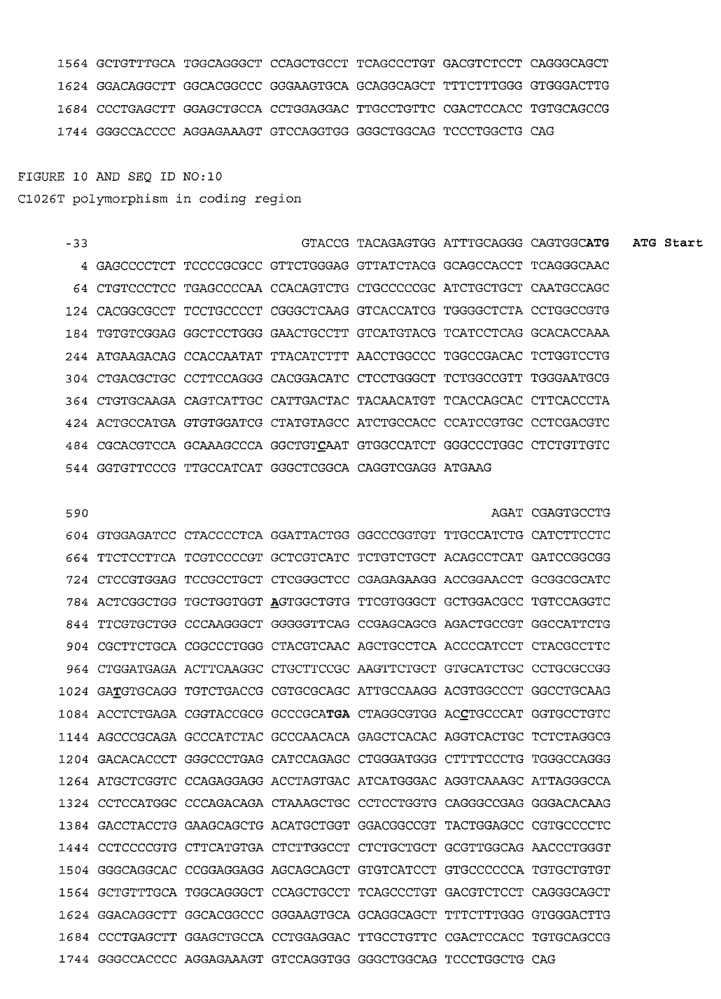

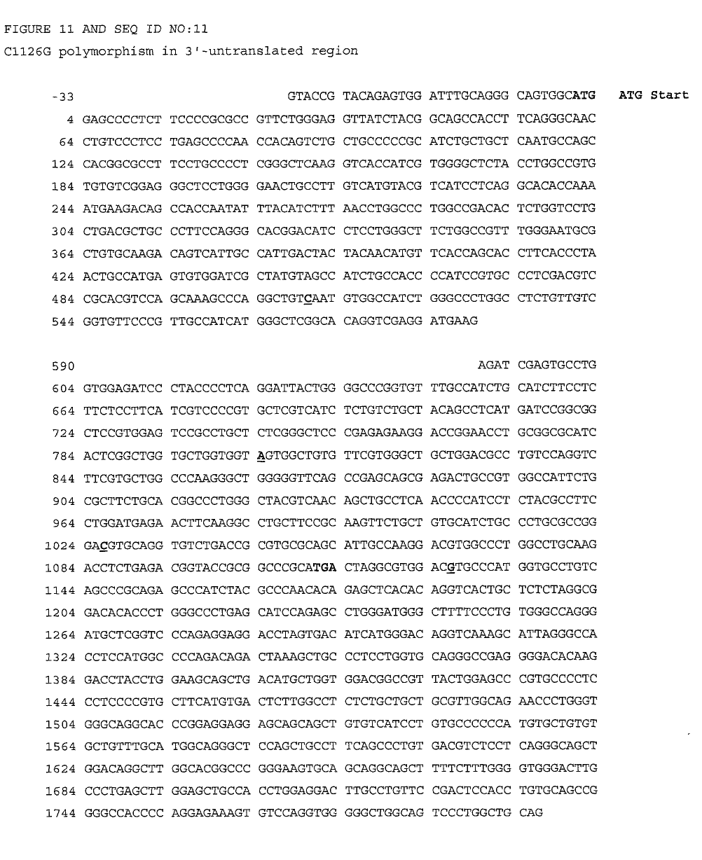

### DETAILED DESCRIPTION OF THE INVENTION

As explained above, the present invention is based upon Applicants' surprising and unexpected discovery of heretofore unknown single nucleotide polymorphisms (SNPs) in the human orphanin FQ/nociceptin receptor gene, along with combinations thereof. Polymorphisms in this gene have not been previously known. Furthermore, Applicants have discovered that more than one polymorphism can be present in either or both alleles of the human orphanin FQ/nociceptin receptor gene in a subject.

In addition, the present invention is based upon Applicants' surprising discovery of molecules of heretofore unknown isolated nucleic acid molecules which encode human orphanin FQ/nociceptin receptors, wherein the DNA sequences comprise one or more polymorphisms as set forth herein.

Furthermore, the present invention is based upon Applicants' surprising and unexpected discovery that the expression of variant alleles of the human orphanin FQ/nociceptin receptor gene comprising a DNA sequence having a variation in SEQ ID NO:1, wherein the variations are: G-46A (G minus 46 A), located in the 5′ untranslated region; GIVS I 135C, located in intron I; GIVS I 250A, located in intron I; GIVS I 251A, located in intron I; C510T, a silent mutation located in the coding region; CIVS III 67T; located in intron III; A804G, a silent mutation located in the coding region; C1026T, a silent mutation located in the coding region; and C1126G, located in the 3′ untranslated region.

The present invention further extends to heretofore unknown polymorphisms of the human orphanin FQ/nociceptin receptor gene that can serve as genetic markers to map the locus of the human orphanin FQ/nociceptin receptor gene.

As noted above, the human orphanin FQ/nociceptin receptor plays important and diverse roles in modulation of the endogenous opioid system, nociception, neurotransmitter release (including dopamine, GABA, noradrenaline, and serotonin), anxiety and stress, learning, memory and cognition, alcohol self-administration, behavioral sensitization to cocaine, drug addiction, opiate withdrawal and tolerance, food intake, immune function, cardiovascular function, renal function, gastrointestinal function, and motor function. As noted herein, reference to the identification of one or more of the polymorphisms described herein and the relationship to physiological response, conditions, disorders, diseases, pathologies, aberrations, and other variations in normal or pathological states relating to the aforementioned physiologic processes is embraced herein as utilities for which the identification of the polymorphisms may be applied. Moreover, the identification of the polymorphisms, whether heterozygous, homozygous, single or multiple polymorphisms in an individual and the linkage of such single or multiple polymorphisms, homozygous or heterozygous, to susceptibility, propensity, therapeutic potential, and other factors are further embraced herein.

The present invention extends to diagnostic methods to determine a subject's increased or decreased susceptibility to at least one disease, including addictive disease. With the results of such methods, targeted prevention methods, early therapeutic intervention, and improved chronic treatment to opioid addiction are set forth herein and encompassed by the present invention. In addition, attending medical professionals of subjects armed with the results of such diagnostic methods can determine whether administration of opioid analgesics is appropriate or whether non-opioid derived analgesics should be administered to the subject. Also, appropriate choice and type of analgesic can be made in treating a subject's pain.

Methods for determining the presence of the one or more polymorphisms may be made using any of a large variety of methods for identifying altered nucleotides present in a nucleic acid sequence, by way of non-limiting examples as conventional DNA sequencing, differential hybridization to biological chip arrays such as an oligonucleotide gelpad microchip, or single nucleotide extension (SNE) on chip arrays such as on oligonucleotide gelpad microchips. These methods are known to one of skill in the art, and are merely exemplified by the following citations: Khrapko K R, Lysov Y P, Khorlin A, Shick V V, Florentiev V L, Mirzabekov A D. 1989. An oligonucleotide hybridization approach to DNA sequencing. FEBS Lett 256:118-122; Khrapko K R, Lysov Y P, Khorlin A A, Ivanov I B, Yershov G M, Vasilenko S L, Florentiev V, Mirzabekov A D, 1991, A method for DNA sequencing by hybridization with oligonucleotide matrix. J DNA sequencing 1: 375-388; Fodor S P A, Read J L, Pirrung M C, Stryer L, Lu A T, Solas, D, 1991, Light directed, spatially addressable parallel chemical synthesis. Science 251:776-773; Southern E M, Maskos U, Elder J K, 1992, Analyzing and comparing nucleic acid sequences by hybridization to arrays of oligonucleotides: evaluation using experimental models, Genomics 13:1008-1017; Chee M, Yang R, Hubbell E, Berno A, Huang X C, Stern D, Winkler J, Lockhart D J, Morris M S, Fodor S P A. 1996. Accessing genetic information with high-density DNA arrays. Science 274:610-614; Hacia J G, Brody L C, Chee M S, Fodor S P A, Collins F. 1996. Detection of heterozygous mutations in BCRA1 using high density oligonucleotide arrays and two colour florescence analysis. Nature Genet 14:44-447; Yershov G, Barsky V, Belgovskiy A, Kirillov E, Kreindlin E, Ivanov I, Parinov S, Guschin D, Drobishev A, Dubiley S, Mirzabekov A. 1996. DNA Analysis and diagnostics on oligonucleotide microchips. Proc Natl Acad Sci USA 93:4913-4918; Shick V V Lebed Y B, Kryukov G V. 1998. Identification of HLA DQA1 alleles by the oligonucleotide microchip method. Mol Biol 32:697-688. Translated from Molekulyarna Biologiya 32:813-822; Wang D G, Fan J-B, Siao C -J, Berno A, Young P, Sapolsky R, Ghandour G, Perkins N, Winchester E, Spencer J, Kruglyak L, Stein L, Hsie L, Topaloglou T, Hubbell E, Robinson E, Mittmann M, Morris MS, Shen N, Kilburn D, Rioux J, Nusbaum C, Rozen S, Hudson T J, Lipschutz R, Chee M, Lander E S. 1998 Large scale identification, mapping and genotyping of single-nucleotide polymorphisms in the human genome. Science 280:1077-1082; Halushka M K, Fan J -B, Bentley K, Hsie L, Shen N, Weder A, Cooper R, Lipshutz R, Chakravarti A. 1999. Patterns of single-nucleotide polymorphisms in candidate genes for blood pressure homeostasis. Nature Genet 22:239-247; Cargill M, Altschuler D, Ireland J, Sklar P, Ardlie K, Patil N, Lane C R, Lim E P, Kalyanaraman N, Nemesh J, Ziaugra L, Friedland L, Rolfe A, Warrington J, Lipshutz R, Daley G Q, Lander E S. 1999. Characterization of single nucleotide polymorphisms in coding regions of human genes. Nature genet 22;231-238; Parinov S, Barsky V, Yershov G, Kirillov E, Timofeev E, Belgovskiy A, Mirzabekov A. 1996. DNA sequencing by hybridization to microchip octa- and decanucleotides extended by stacked pentanucleotides. Nucleic Acids Res 24:2998-3004; Guschin D, Yershof G, Zaslavsky A, Gemmell A, Shick V, Proudnikov V, Arenkov P, Mirzabekov A. 1997. Manual manufacturing of oligonucleotide, DNA and protein microchips. Anal Biochem 250:203-211; Drobyshev A, Mologina M. Shik V, Pobedimskaya D, Yershov G, Mirzabekov A. 1997. Sequence analysis by hybridization with oligonucleotide microchip: Identification of b-thalassemia mutations. Gene 188:45-52; Syvänen A -C, Aalto-Setälä K, Harju L, Kontula K, SØderlund H. 1990. A primer-guided nucleotide incorporation assay in the genotyping of apolipoprotein E. Genomics 8:684-692; Pastinen T, Kurg A, Metspalu A, Peltonen L, Syvänen A -C. 1997. Minisequencing: A specific tool for DNA analysis and diagnostics on oligonucleotide arrays. Genome res 7:606-614; Pastinen T, Perola M, Niini P, Terwilliger J, Salomaa V, Vartiainen E, Peltonen L, Syvänen A-C. 1998. Array-based multiplex analysis of candidate gene reveals two independent and additive genetic risk factors for myocardial infarction in the Finnish population. Hum Mol Genet 7:1453-1462; Dubiley S, Kirillov E, Mirzabekov A. 1999. Polymorphism analysis and gene detection by minisequencing on an array of gel-immobilized primers. Nucleic Acids Res 27:el9; and Syvänen A-C. 1999. From gels to chips: “Minisequencing” primer extension analysis of point mutations and single nucleotide polymorphisms. Hum Mutat 13: 1-10. Such citations are not intended to be limiting but merely exemplary of the various methods available for detecting one or more of the polymorphisms described herein.

Also, the present invention extends to methods of determining a subject's increased or decreased susceptibility to pain and response to analgesics, and using that information when prescribing analgesics to the subject.

The present invention further extends to variant alleles of the human orphanin FQ/nociceptin receptor gene comprising a DNA sequence comprising one or more heretofore unknown polymorphisms, G-46A, GIVS I 135C, GIVS I 250A, GIVS I 251A, C510T, CIVS III 67T, A804G, C1026T, or C1126G.

Consequently, an initial aspect of the present invention involves isolation of heretofore unknown variant alleles of the human orphanin FQ/nociceptin receptor gene. As used herein, the term “gene” refers to an assembly of nucleotides that encode a polypeptide, and includes cDNA and genomic DNA nucleic acids.

Furthermore, in accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning. A Laboratory Manual, Second Edition (1989) Cold Spring Harbor Laboratory Press, Cold Spring Harbor, New York (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid hybridization [B. D. Hames & S. J. Higgins eds. (1985)]; Transcription And Translation [B. D. Hames & S. J. Higgins, eds. (1984)]; Animal Cell Culture [R. I. Freshney, ed. (1986)]; Immobilized Cells And Enzymes [IRL Press, (1986)]; B. Perbal, A Practical Guide To Molecular Cloning (1984); F. M. Ausubel et al. (eds.), Current Protocols in Molecular Biology, John Wiley & Sons, Inc. (1994).

Therefore, if appearing herein, the following terms shall have the definitions set out below.

A “vector” is a replicon, such as plasmid, phage or cosmid, to which another DNA segment may be attached so as to bring about the replication of the attached segment. A “replicon” is any genetic element (e.g., plasmid, chromosome, virus) that functions as an autonomous unit of DNA replication in vivo, i.e., capable of replication under its own control.

A “cassette” refers to a segment of DNA that can be inserted into a vector at specific restriction sites. The segment of DNA encodes a polypeptide of interest, and the cassette and restriction sites are designed to ensure insertion of the cassette in the proper reading frame for transcription and translation.

A cell has been “transfected” by exogenous or heterologous DNA when such DNA has been introduced inside the cell. A cell has been “transformed” by exogenous or heterologous DNA when the transfected DNA effects a phenotypic change. Preferably, the transforming DNA should be integrated (covalently linked) into chromosomal DNA making up the genome of the cell.

“Heterologous” DNA refers to DNA not naturally located in the cell, or in a chromosomal site of the cell. Preferably, the heterologous DNA includes a gene foreign to the cell.

A “nucleic acid molecule” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA). A “recombinant DNA molecule” is a DNA molecule that has undergone a molecular biological manipulation.

A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength (see Sambrook et al., supra). The conditions of temperature and ionic strength determine the “stringency” of the hybridization. Polynucleotides capable of discriminating between the wild-type and polymorphic alleles of the invention (“selectively hybridizable”) may be prepared, and the conditions under which such polynucleotides selectively hybridize with the polymorphisms of the invention, may be achieved following guidance provided in the art, such as described by Conner et al., 1983, Proc. Nat. Acad. Sci. U.S.A. 80:278-82;Yershov et al., 1996, Proc. Nat. Acad. Sci. U.S.A. 93:4913-18; Drobyshev et al., 1997, Gene 188:45-52; and Chee et al., 1996, Science 274:610-614. Selectively hybridizable reporting polynucleotides such as molecular beacons are also well known in the art.

For preliminary screening for homologous nucleic acids, low stringency hybridization conditions, corresponding to a T. of 55°, can be used, e.g., 5× SSC, 0.1% SDS, 0.25% milk, and no formamide; or 30% formamide, 5× SSC, 0.5% SDS). Moderate stringency hybridization conditions correspond to a higher Tₘ, e.g., 40% formamide, with 5× or 6× SSC. High stringency hybridization conditions correspond to the highest Tₘ, e.g., 50% formamide, 5× or 6× SSC. Hybridization requires that the two nucleic acids contain complementary sequences, although depending on the stringency of the hybridization, mismatches between bases are possible. The appropriate stringency for selectively hybridizing nucleic acids depends on the length of the nucleic acids and the degree of complementation, variables well known in the art. The greater the degree of similarity or homology between two nucleotide sequences, the greater the value of Tₘ for hybrids of nucleic acids having those sequences. The relative stability (corresponding to higher Tₘ of nucleic acid hybridizations decreases in the following order: RNA:RNA, DNA:RNA, DNA:DNA. For hybrids of greater than 100 nucleotides in length, equations for calculating Tₘ have been derived (see Sambrook et al., supra, 9.50-0.51). For hybridization with shorter nucleic acids, i.e., oligonucleotides, the position of mismatches becomes more important, and the length of the oligonucleotide determines its specificity (see Sambrook et al., supra, 11.7-11.8). Preferably a minimum length for a selectively hybridizable nucleic acid is at least about 10 nucleotides; preferably at least about 20 nucleotides; and more preferably the length is at least about 30 nucleotides; and most preferably 40 nucleotides. As noted above, the skilled artisan will be guided by the teachings in the art on selecting the length of a polynucleotide or nucleic acid sequence, the position(s) of the variant nucleotide(s), and the conditions and instrumentation to selectively identify nucleic acid sequences comprising one or more of the polymorphisms as described herein.

In a specific embodiment, the term “standard hybridization conditions” refers to a Tₘ of 55° C., and utilizes conditions as set forth above. In a preferred embodiment, the Tₘ is 60° C.; in a more preferred embodiment, the Tₘ is 65° C.

“Homologous recombination” refers to the insertion of a foreign DNA sequence of a vector in a chromosome. Preferably, the vector targets a specific chromosomal site for homologous recombination. For specific homologous recombination, the vector will contain sufficiently long regions of homology to sequences of the chromosome to allow complementary binding and incorporation of the vector into the chromosome. Longer regions of homology, and greater degrees of sequence similarity, may increase the efficiency of homologous recombination.

A DNA “coding sequence” is a double-stranded DNA sequence which is transcribed and translated into a polypeptide in a cell in vitro or in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from eukaryotic mRNA, genomic DNA sequences from eukaryotic (e.g., mammalian) DNA, and even synthetic DNA sequences. If the coding sequence is intended for expression in a eukaryotic cell, a polyadenylation signal and transcription termination sequence will usually be located 3′ to the coding sequence.

Transcriptional and translational control sequences are DNA regulatory sequences, such as promoters, enhancers, terminators, and the like, that provide for the expression of a coding sequence in a host cell. In eukaryotic cells, polyadenylation signals are control sequences.

A “promoter sequence” or “promoter” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase.

A coding sequence is “under the control” of transcriptional and translational control sequences in a cell when RNA polymerase transcribes the coding sequence into mRNA, which is then trans-RNA spliced and translated into the protein encoded by the coding sequence.

A coding sequence is “operatively associated with” a transcriptional and translational control sequences, such as a promoter for example, when RNA polymerase transcribes the coding sequence into mRNA, which in turn is translated into a protein encoding by the coding sequence.

A “signal sequence” is included at the beginning of the coding sequence of a protein to be expressed on the surface of a cell. This sequence encodes a signal peptide, N-terminal to the mature polypeptide, that directs the host cell to translocate the polypeptide. The term “translocation signal sequence” is used herein to refer to this sort of signal sequence. Translocation signal sequences can be found associated with a variety of proteins native to eukaryotes and prokaryotes, and are often functional in both types of organisms.

An “expression control sequence” is a DNA sequence that controls and regulates the transcription and translation of another DNA sequence. A coding sequence is “under the control” of transcriptional and translational control sequences in a cell when RNA polymerase transcribes the coding sequence into mRNA, which is then translated into the protein encoded by the coding sequence.

The term “primer” as used herein refers to an oligonucleotide, whether occurring naturally as in a purified restriction digest or produced synthetically, which is capable of acting as a point of initiation of synthesis when placed under conditions in which synthesis of a primer extension product, which is complementary to a nucleic acid strand, is induced, i.e., in the presence of nucleotides and an inducing agent such as a DNA polymerase and at a suitable temperature and pH. The primer may be either single-stranded or double-stranded and must be sufficiently long to prime the synthesis of the desired extension product in the presence of the inducing agent. The exact length of the primer will depend upon many factors, including temperature, source of primer and use of the method. For example, for diagnostic applications, depending on the complexity of the target sequence, the oligonucleotide primer typically contains 15-25 or more nucleotides, although it may contain fewer nucleotides.

The primers herein are selected to be “substantially” complementary to different strands of a particular target DNA sequence. This means that the primers must be sufficiently complementary to selectively hybridize with their respective strands. Therefore, the primer sequence need not reflect the exact sequence of the template. For example, a non-complementary nucleotide fragment may be attached to the 5′ end of the primer, with the remainder of the primer sequence being complementary to the strand. Alternatively, non-complementary bases or longer sequences can be interspersed into the primer, provided that the primer sequence has sufficient complementarity with the sequence of the strand to selectively hybridize therewith and thereby form the template for the synthesis of the extension product.

A cell has been “transformed” by exogenous or heterologous DNA when such DNA has been introduced inside the cell. The transforming DNA may or may not be integrated (covalently linked) into chromosomal DNA making up the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA. A “clone” is a population of cells derived from a single cell or common ancestor by mitosis. A “cell line” is a clone of a primary cell that is capable of stable growth in vitro for many generations.

The phrase “expected to be indicative” is used herein to refer to the correlation between the identity of the allelic variation(s) in an individual and the susceptibility of an individual to addictive disease, sensitivity to pain and analgesics, therapeutic effectiveness of analgesics, and other physiological manifestations described herein related to the function of the orphanin FQ/nociceptin receptor, such as but not limited to the endogenous opioid system, nociception, neurotransmitter release (including dopamine, GABA, noradrenaline, and serotonin), anxiety and stress, learning, memory and cognition, alcohol self-administration, behavioral sensitization to cocaine, drug addiction, opiate withdrawal and tolerance, food intake, immune function, cardiovascular function, renal function, gastrointestinal function, and motor function. Expected correlations of orphanin FQ/nociceptin receptor alleles and susceptibility to various conditions may be increased susceptibility or decreased susceptibility.

As explained above, within the scope of the present invention are DNA sequences encoding variant alleles of a human orphanin FQ/nociceptin receptor gene of the present invention, which comprise at least one variation in the predominant or “most common” allele of the human orphanin FQ/nociceptin receptor gene. The most common allele comprises a DNA sequence of SEQ ID NO:1, and variations in the most common allele comprise G-46A, GIVS I 135C, GIVS I 250A, GIVS I 251A, C510T, CIVS III 67T, A804G, C1026T, or C1126G.

As used herein, the term “sequence homology” in all its grammatical forms refers to the relationship between proteins that possess a “common evolutionary origin,” including proteins from superfamilies (e.g., the immunoglobulin superfamily) and homologous proteins from different species (e.g., myosin light chain, etc.) (Reeck et al., 1987, Cell 50:667).

Accordingly, the term “sequence similarity” in all its grammatical forms refers to the degree of identity or correspondence between nucleic acid or amino acid sequences of proteins that do not share a common evolutionary origin (see Reeck et al., supra). However, in common usage and in the instant application, the term “homologous,” when modified with an adverb such as “highly,” may refer to sequence similarity and not a common evolutionary origin.

In a specific embodiment, two DNA sequences are “substantially homologous” or “substantially similar” when at least about 50% (preferably at least about 75%, and most preferably at least about 90 or 95%) of the nucleotides match over the defined length of the DNA sequences. Sequences that are substantially homologous can be identified by comparing the sequences using standard software available in sequence data banks, or in a Southern hybridization experiment under, for example, stringent conditions as defined for that particular system. Defining appropriate hybridization conditions is within the skill of the art. See, e.g., Maniatis et al., supra; DNA Cloning, Vols. I & II, supra; Nucleic Acid Hybridization, supra.

The term “corresponding to” is used herein to refer to similar or homologous sequences, whether the exact position is identical or different from the molecule to which the similarity or homology is measured. Thus, the term “corresponding to” refers to the sequence similarity, and not the numbering of the amino acid residues or nucleotide bases.

A variant allele of the human orphanin FQ/nociceptin receptor gene of the present invention, whether genomic DNA or cDNA, can be isolated from any source, particularly from a human cDNA or genomic library. Methods for obtaining an allele of a human orphanin FQ/nociceptin receptor gene, variants thereof, or the most common, are well known in the art, as described above (see, e.g., Sambrook et al., 1989, supra).

Accordingly, any human cell potentially can serve as the nucleic acid source for the molecular cloning of a variant allele of the human orphanin FQ/nociceptin receptor gene of the present invention, or a nucleic acid molecule selectively hybridizable to a variant allele of a human orphanin FQ/nociceptin receptor gene of the present invention. The DNA may be obtained by standard procedures known in the art from cloned DNA (e.g., a DNA “library”), and preferably is obtained from a cDNA library prepared from tissues with high level expression of a human orphanin FQ/nociceptin receptor protein, by chemical synthesis, by cDNA cloning, or by the cloning of genomic DNA, or fragments thereof, purified from the desired cell (See, for example, Sambrook et al., 1989, supra; Glover, D. M. (ed.), 1985, DNA Cloning: A Practical Approach, MRL Press, Ltd., Oxford, U.K. Vol. 1, II). Clones derived from genomic DNA may contain regulatory and intron DNA regions in addition to coding regions; clones derived from cDNA will not contain intron sequences. Whatever the source, an allele of a human orphanin FQ/nociceptin receptor gene of the present invention should be molecularly cloned into a suitable vector for propagation.

In the molecular cloning of a human orphanin FQ/nociceptin receptor gene of the present invention, DNA fragments are generated, some of which will encode an allele. The DNA may be cleaved at specific sites using various restriction enzymes. Alternatively, one may use DNAse in the presence of manganese to fragment the DNA, or the DNA can be physically sheared, as for example, by sonication. The linear DNA fragments can then be separated according to size by standard techniques, including but not limited to, agarose and polyacrylamide gel electrophoresis and column chromatography.

Once the DNA fragments are generated, identification of the specific DNA fragment containing an allele of a human orphanin FQ/nociceptin receptor of the present invention may be accomplished in a number of ways. For example, if an amount of a portion of an allele of a human orphanin FQ/nociceptin receptor gene, or its specific RNA, or a fragment thereof, is available and can be purified and labeled, the generated DNA fragments may be screened by nucleic acid hybridization to the labeled probe (Benton and Davis, 1977, Science 196:180; Grunstein and Hogness, 1975, Proc. Natl. Acad. Sci. U.S.A. 72:3961). For example, a set of oligonucleotides corresponding to the partial amino acid sequence information obtained for a human orphanin FQ/nociceptin receptor protein can be prepared and used as probes for DNA encoding a variant allele of a human orphanin FQ/nociceptin receptor gene of the present invention, as was done in a specific example, infra, or as primers for cDNA or mRNA (e.g., in combination with a poly-T primer for RT-PCR). Preferably, a fragment is selected that is highly unique to a variant allele of the human orphanin FQ/nociceptin receptor gene of the invention. Those DNA fragments with substantial homology to the probe will selectively hybridize. As noted above, the greater the degree of homology, the more stringent hybridization conditions can be used.

An allele of a human orphanin FQ/nociceptin receptor gene of the present invention can also be identified by mRNA selection, i.e., by nucleic acid hybridization followed by in vitro translation. In this procedure, nucleotide fragments are used to isolate complementary mRNAs by hybridization. Such DNA fragments may represent available, purified DNA of an allele of a human orphanin FQ/nociceptin receptor gene of the present invention, or may be synthetic oligonucleotides designed from the partial amino acid sequence information. Immunoprecipitation analysis or functional assays of the in vitro translation products of the products of the isolated mRNAs identifies the mRNA and, therefore, the complementary DNA fragments, that contain the desired sequences.

A labeled cDNA of an allele of a human orphanin FQ/nociceptin receptor gene of the present invention, or fragments thereof, or a nucleic acid selectively hybridizable to an allele of a human orphanin FQ/nociceptin receptor gene of the present invention, can be synthesized using sequences set forth herein. The radiolabeled mRNA or cDNA may then be used as a probe to identify homologous DNA fragments from among other genomic DNA fragments. Suitable labels include enzymes, radioactive isotopes, fluorophores (e.g., fluorescein isothiocyanate (FITC), phycoerythrin (PE), Texas red (TR), rhodamine, free or chelated lanthanide series salts, especially Eu³⁺, to name a few fluorophores), chromophores, radioisotopes, chelating agents, dyes, colloidal gold, latex particles, ligands (e.g., biotin), and chemiluminescent agents. When a control marker is employed, the same or different labels may be used for the receptor and control marker. As noted above, molecular beacons capable of identifying the polymorphisms of the invention are embraced herein.

In the instance where a radioactive label, such as the isotopes ³H, ¹⁴C, ³²p, ³⁵S, ³⁶C1, ⁵¹Cr, ⁵⁷Co, ⁵⁸Co, ⁵⁹Fe, ⁹⁰Y, ^{125I,} ¹³¹I, and ¹⁸⁶Re are used, known currently available counting procedures may be utilized. In the instance where the label is an enzyme, detection may be accomplished by any of the presently utilized calorimetric, spectrophotometric, fluorospectrophotometric, amperometric or gasometric techniques known in the art.

Direct labels are one example of labels which can be used according to the present invention. A direct label has been defined as an entity, which in its natural state, is readily visible, either to the naked eye, or with the aid of an optical filter and/or applied stimulation, e.g., U.V. light to promote fluorescence. Among examples of colored labels, which can be used according to the present invention, include metallic sol particles, for example, gold sol particles such as those described by Leuvering (U.S. Pat. No. 4,313,734); dye sol particles such as described by Gribnau et al. (U.S. Pat. No. 4,373,932) and May et al. (WO 88/08534); dyed latex such as described by May, supra, Snyder (EP-a 0 280 559 and 0 281 327); or dyes encapsulated in liposomes as described by Campbell et al. (U.S. Pat. No. 4,703,017). Other direct labels include a radionucleotide, a fluorescent moiety or a luminescent moiety. In addition to these direct labeling devices, indirect labels comprising enzymes can also be used according to the present invention. Various types of enzyme linked immunoassays are well known in the art, for example, alkaline phosphatase and horseradish peroxidase, lysozyme, glucose-6-phosphate dehydrogenase, lactate dehydrogenase, urease, these and others have been discussed in detail by Eva Engvall in Enzyme Immunoassay ELISA and EMIT in Methods in Enzymology, 70. 419-439, 1980 and in U.S. Pat. No. 4,857,453.

Other labels for use in the invention include magnetic beads or magnetic resonance imaging labels.

### Cloning Vectors

The present invention also relates to cloning vectors comprising variant alleles of a human orphanin FQ/nociceptin receptor gene of the present invention, and an origin of replication. For purposes of this Application, an “origin of replication refers to those DNA sequences that participate in DNA synthesis.

As explained above, in an embodiment of the present invention, variant alleles of a human orphanin FQ/nociceptin receptor gene of the present invention comprise a DNA sequence having at least one variation in the most common allele of a human orphanin FQ/nociceptin receptor gene comprising a DNA sequence of SEQ ID NO:1, wherein the variation comprises G-46A, GIVS I 135C, GIVS I 250A, GIVS I 251A, C510T, CIVS III 67T, A804G, C1026T, C1126G, or combinations thereof.

Furthermore, an isolated variant allele of a human orphanin FQ/nociceptin receptor gene of the present invention, or isolated nucleic acid molecules selectively hybridizable to an isolated variant allele of a human orphanin FQ/nociceptin receptor gene of the present invention, can be inserted into an appropriate cloning vector in order to produce multiple copies of the variant allele or isolated nucleic acid molecule. A large number of vector-host systems known in the art may be used. Possible vectors include, but are not limited to, plasmids or modified viruses. The vector system used however must be compatible with the host cell used. Examples of vectors include having applications herein, but are not limited to E. coli, bacteriophages such as lambda derivatives, or plasmids such as pBR322 derivatives or pUC plasmid derivatives, e.g., pGEX vectors, pmal-c, pFLAG, etc. The insertion into a cloning vector can, for example, be accomplished by ligating a variant allele of the human orphanin FQ/nociceptin receptor gene of the present invention, or an isolated nucleic acid selectively hybridizable thereto, into a cloning vector which has complementary cohesive termini. However, if the complementary restriction sites used to fragment the variant allele or isolated nucleic acid selectively hybridizable thereto are not present in the cloning vector, the ends of the variant allele or the isolated nucleic acid molecule selectively hybridizable thereto may be enzymatically modified. Alternatively, any site desired may be produced by ligating nucleotide sequences (linkers) onto the DNA termini; these ligated linkers may comprise specific chemically synthesized oligonucleotides encoding restriction endonuclease recognition sequences. Such recombinant molecules can then be introduced into host cells via transformation, transfection, infection, electroporation, etc., so that many copies of a variant allele of a human orphanin FQ/nociceptin receptor gene of the present invention, or an isolated nucleic acid molecule selectively hybridizable thereto, can be generated. Preferably, the cloned isolated variant is contained on a shuttle vector plasmid, which provides for expansion in a cloning cell, e.g., E. coli, and facile purification for subsequent insertion into an appropriate expression cell line, if such is desired. For example, a shuttle vector, which is a vector that can replicate in more than one type of organism, can be prepared for replication in both E. coli and Saceharomyces cerevisiae by linking sequences from an E. coli plasmid with sequences from the yeast 2μ plasmid.

In an alternative method an isolated variant allele of a human orphanin FQ/nociceptin receptor gene of the present invention or an isolated nucleic acid molecule selectively hybridizable thereto may be identified and isolated after insertion into a suitable cloning vector in a “shot gun” approach. Enrichment for a variant allele, for example, by size fractionation, can be done before insertion into the cloning vector.

### Expression Vectors

As stated above, the present invention extends to an isolated variant allele of a human orphanin FQ/nociceptin receptor gene, comprising a DNA sequence having at least one variation in the DNA sequence of the predominant or “most common” allele of the human orphanin FQ/nociceptin receptor gene comprising a DNA sequence of SEQ ID NO:1 wherein the variations comprise G-46A, GIVS I 135C, GIVS I 250A, GIVS I 251A, C510T, CIVS III 67T, A804G, C1026T, C126G, or combinations thereof.

Variant alleles of the present invention, along with isolated nucleic acid molecules selectively hybridizable to such variant alleles, can be inserted into an appropriate expression vector, i.e., a vector which contains the necessary elements for the transcription and translation of the inserted protein-coding sequence. Thus, a variant allele of the present invention, or an isolated nucleic acid molecule selectively hybridizable to a variant allele of the present invention, is operatively associated with a promoter in an expression vector of the invention. A DNA sequence is “operatively associated” to an expression control sequence, such as a promoter, when the expression control sequence controls and regulates the transcription and translation of that DNA sequence. The term “operatively associated” includes having an appropriate start signal (e.g., ATG) in front of the DNA sequence to be expressed and maintaining the correct reading frame to permit expression of the DNA sequence under the control of the expression control sequence and production of the desired product encoded by the DNA sequence. If a variant allele of the present invention, or an isolated nucleic acid selectively hybridizable thereto does not contain an appropriate start signal, such a start signal can be inserted into the expression vector in front of (5′ of) the molecule.

Both cDNA and genomic sequences can be cloned and expressed under control of such regulatory sequences. An expression vector also preferably includes a replication origin.

The necessary transcriptional and translational signals can be provided on a recombinant expression vector, or they may be supplied by an allele comprising a human orphanin FQ/nociceptin receptor gene.

Potential host-vector systems include but are not limited to mammalian cell systems infected with virus (e.g., vaccinia virus, adenovirus, etc.); insect cell systems infected with virus (e.g., baculovirus); microorganisms such as yeast containing yeast vectors; or bacteria transformed with bacteriophage, DNA, plasmid DNA, or cosmid DNA. The expression elements of vectors vary in their strengths and specificities. Depending on the host-vector system utilized, any one of a number of suitable transcription and translation elements may be used.

A variant allele of a human orphanin FQ/nociceptin receptor gene of the present invention or an isolated nucleic acid molecule selectively hybridizable thereto may be expressed chromosomally, after integration of the coding sequence by recombination. In this regard, any of a number of amplification systems may be used to achieve high levels of stable gene expression (See Sambrook et al., 1989, supra).

A unicellular host transformed or transfected with an expression vector of the present invention is cultured in an appropriate cell culture medium that provides for expression by the unicellular host of the variant allele, or isolated nucleic acid selectively hybridizable thereto.

Any of the methods previously described for the insertion of DNA fragments into a cloning vector may be used to construct expression vectors of the present invention. These methods may include in vitro recombinant DNA and synthetic techniques and in vivo recombination (genetic recombination).

Expression of a variant allele of a human orphanin FQ/nociceptin receptor gene of the present invention or an isolated nucleic acid molecule selectively hybridizable to a variant allele of a human orphanin FQ/nociceptin receptor gene, may be controlled by any promoter/enhancer element known in the art, but these regulatory elements must be functional in the host selected for expression. Promoters which may be used to control expression include, but are not limited to, the SV40 early promoter region (Benoist and Chambon, 1981, Nature 290:304-310), the promoter contained in the 3′ long terminal repeat of Rous sarcoma virus (Yamamoto, et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al., 1982, Nature 296:39-42); prokaryotic expression vectors such as the β-lactamase promoter (Villa-Kamaroff, et al., 1978, Proc. Natl. Acad. Sci. U.S.A. 75:3727-3731), or the tac promoter (DeBoer, et al., 1983, Proc. Natl. Acad. Sci. U.S.A. 80:21-25); see also “Useful proteins from recombinant bacteria” in Scientific American, 1980, 242:74-94; promoter elements from yeast or other fungi such as the Gal 4 promoter, the ADC (alcohol dehydrogenase) promoter, PGK (phosphoglycerol kinase) promoter, alkaline phosphatase promoter; and the animal transcriptional control regions, which exhibit tissue specificity and have been utilized in transgenic animals: elastase I gene control region which is active in pancreatic acinar cells (Swift et al., 1984, Cell 38:639-646; Ornitz et al., 1986, Cold Spring Harbor Symp. Quant. Biol. 50:399-409; MacDonald, 1987, Hepatology 7:425-515); insulin gene control region which is active in pancreatic beta cells (Hanahan, 1985, Nature 315:115-122), immunoglobulin gene control region which is active in lymphoid cells (Grosschedl et al., 1984, Cell 38:647-658; Adames et al., 1985, Nature 318:533-538; Alexander et al., 1987, Mol. Cell. Biol. 7:1436-1444), mouse mammary tumor virus control region which is active in testicular, breast, lymphoid and mast cells (Leder et al., 1986, Cell 45:485-495), albumin gene control region which is active in liver (Pinkert et al., 1987, Genes and Devel. 1:268-276), alpha-fetoprotein gene control region which is active in liver (Krumlauf et al., 1985, Mol. Cell. Biol. 5:1639-1648; Hammer et al., 1987, Science 235:53-58), alpha 1-antitrypsin gene control region which is active in the liver (Kelsey et al., 1987, Genes and Devel. 1: 161-171), beta-globin gene control region which is active in myeloid cells (Mogram et al., 1985, Nature 315:338-340; Kollias et al., 1986, Cell 46:89-94), myelin basic protein gene control region which is active in oligodendrocyte cells in the brain (Readhead et al., 1987, Cell 48:703-712), myosin light chain-2 gene control region which is active in skeletal muscle (Sani, 1985, Nature 314:283-286), and gonadal releasing hormone gene control region which is active in the hypothalamus (Mason et al., 1986, Science 234:1372-1378).

Moreover, expression vectors comprising a variant allele of a human orphanin FQ/nociceptin receptor gene of the present invention, or an isolated nucleic acid molecule selectively hybridizable thereto, can be identified by four general approaches: (a) PCR amplification of the desired plasmid DNA or specific mRNA, (b) nucleic acid hybridization, (c) presence or absence of selection marker gene functions, and (d) expression of inserted sequences. In the first approach, the variant allele or isolated nucleic acid molecule selectively hybridizable thereto can be amplified by PCR to provide for detection of the amplified product. This includes a molecular beacon approach to identifying the polymorphisms herein. In the second approach, the presence of a foreign gene inserted into an expression vector of the present invention can be detected by nucleic acid hybridization using probes comprising sequences that are homologous to an inserted marker gene. In the third approach, the recombinant vector/host system can be identified and selected based upon the presence or absence of certain “selection marker” gene functions (e.g., β-galactosidase activity, thymidine kinase activity, resistance to antibiotics, transformation phenotype, occlusion body formation in baculovirus, etc.) caused by the insertion of foreign genes in the vector. In yet another example, if an isolated variant allele of a human orphanin FQ/nociceptin receptor gene of the present invention, or an isolated nucleic acid molecule selectively hybridizable thereto, is inserted within the “selection marker” gene sequence of the vector, recombinants containing the insert can be identified by the absence of the inserted gene function. In the fourth approach, recombinant expression vectors can be identified by assaying for the activity, biochemical, or immunological characteristics of the gene product expressed by the recombinant, provided that the expressed protein assumes a functionally active conformation.

Naturally, the present invention extends to a method of producing a human orphanin FQ/nociceptin receptor from the polymorphic variants described herein. Although the variants described herein are “silent,” as they do not alter the amino acid sequence of the orphanin FQ/nociceptin gene product (i.e., the receptor), the methods herein may be used to determine altered levels of gene expression as a consequence of the presence of one or more of the polymorphisms described herein. An example of such a method comprises the steps of culturing a unicellular host transformed or transfected with an expression vector comprising a variant allele of a human orphanin FQ/nociceptin receptor gene comprising a DNA sequence having a variation in SEQ ID NO:1, wherein the variant allele which is operatively associated with a promoter. The transformed or transfected unicellular host is then cultured under conditions that provide for expression of the variant allele of the human orphanin FQ/nociceptin receptor gene, and the expression product is recovered from the unicellular host.

Another example involves culturing a unicellular host transformed or transfected with an isolated nucleic acid molecule selectively hybridizable to a variant allele of a human orphanin FQ/nociceptin receptor gene comprising a DNA sequence having at least one variation in SEQ ID NO:1, wherein the isolated nucleic acid molecule is operatively associated with a promoter. The variant human orphanin FQ/nociceptin receptor is then recovered from the host.

A wide variety of unicellular host/expression vector combinations may be employed in expressing the DNA sequences of this invention. Useful expression vectors, for example, may consist of segments of chromosomal, non-chromosomal and synthetic DNA sequences. Suitable vectors include derivatives of SV40 and known bacterial plasmids, e.g., E. coli plasmids col E1, pCR1, pBR322, pMal-C2, pET, pGEX (Smith et al., 1988, Gene 67:31-40), pMB9 and their derivatives, plasmids such as RP4; phage DNAs, e.g., the numerous derivatives of phage λ, e.g., NM989, and other phage DNA, e.g., M13 and filamentous single stranded phage DNA; yeast plasmids such as the 2μ plasmid or derivatives thereof; vectors useful in eukaryotic cells, such as vectors useful in insect or mammalian cells; vectors derived from combinations of plasmids and phage DNAs, such as plasmids that have been modified to employ phage DNA or other expression control sequences; and the like.

For example, in a baculovirus expression systems, both non-fusion transfer vectors, such as but not limited to pVL941 (BamH1 cloning site; Summers), pVL1393 (BamH1, SmaI, XbaI, EcoR1, NotI, XmaIII, BglII, and PstI cloning site; Invitrogen), pVL1392 (BglII, PstI, NotI, XmaIII, EcoRI, XbaI, SmaI, and BamH1 cloning site; Summers and Invitrogen), and pBlueBacIII (BamH1, BglII, PstI, NcoI, and HindIII cloning site, with blue/white recombinant screening possible; Invitrogen), and fusion transfer vectors, such as but not limited to pAc700 (BamH1 and KpnI cloning site, in which the BamH1 recognition site begins with the initiation codon; Summers), pAc701 and pAc702 (same as pAc700, with different reading frames), pAc360 (BamH1 cloning site 36 base pairs downstream of a polyhedrin initiation codon; Invitrogen(195)), and pBlueBacHisA, B, C (three different reading frames, with BamH1, BglII, PstI, NcoI, and HindIII cloning site, an N-terminal peptide for ProBond purification, and blue/white recombinant screening of plaques; Invitrogen (220)) can be used.

Mammalian expression vectors contemplated for use in the invention include vectors with inducible promoters, such as the dihydrofolate reductase (DHFR) promoter, e.g., any expression vector with a DHFR expression vector, or a DHFR/methotrexate co-amplification vector, such as pED PstI, SalI, SbaI, SmaI, and EcoRI cloning site, with the vector expressing both the cloned gene and DHFR; see Kaufman, Current Protocols in Molecular Biology, 16.12 (1991).

Alternatively, a glutamine synthetase/methionine sulfoximine co-amplification vector, such as pEE14 (HindIII, XbaI, SmaI, SbaI, EcoRI, and BclI cloning site, in which the vector expresses glutamine synthase and the cloned gene; Celltech). In another embodiment, a vector that directs episomal expression under control of Epstein Barr Virus (EBV) can be used, such as pREP4 (BamH1, SfiI, Xhol, NotI, NheI, HindIII, NheI, PvuII, and KpnI cloning site, constitutive RSV-LTR promoter, hygromycin selectable marker; Invitrogen), pCEP4 (BamH1, SfiI, XhoI, NotI, NheI, HindIII, NheI, PvuII, and KpnI cloning site, constitutive hCMV immediate early gene, hygromycin selectable marker; Invitrogen), pMEP4 (KpnI, PvuI, NheI, HindIII, NotI, Xhol, SfiI, BamH1 cloning site, inducible metallothionein IIa gene promoter, hygromycin selectable marker: Invitrogen), pREP8 (BamH1, XhoI, NotI, HindIII, NheI, and KpnI cloning site, RSV-LTR promoter, histidinol selectable marker; Invitrogen), pREP9 (KpnI, NheI, HindIII, NotI, Xhol, SfiI, and BamH1 cloning site, RSV-LTR promoter, G418 selectable marker; Invitrogen), and pEBVHis (RSV-LTR promoter, hygromycin selectable marker, N-terminal peptide purifiable via ProBond resin and cleaved by enterokinase; Invitrogen). Selectable mammalian expression vectors for use in the invention include pRc/CMV (HindIII, BstXI, NotI, ShaI, and ApaI cloning site, G418 selection; Invitrogen), pRc/RSV (HindIII, SpeI, BstXI, NotI, XbaI cloning site, G418 selection; Invitrogen), and others. Vaccinia virus mammalian expression vectors (see, Kaufman, 1991, supra) for use according to the invention include but are not limited to pSC11 (SmaI cloning site, TK- and β-gal selection), pMJ601 (SalI, SmaI, AflI, NarI, BspMII, BamHI, ApaI, NheI, SacII, KpnI, and HindIII cloning site; TK- and β-gal selection), and pTKgptF IS (EcoRI, PstI, SalI, AccI, HindIII, SbaI, BamHI, and Hpa cloning site, TK or XPRT selection).

Yeast expression systems can also be used according to the invention to produce a variant human orphanin FQ/nociceptin receptor or the present invention. For example, the non-fusion pYES2 vector (XbaI, SphI, ShoI, NotI, GstXI, EcoRI, BstXI, BamH1, SacI, Kpn1, and HindIII cloning sit; Invitrogen) or the fusion pYESHisA, B, C (XbaI, SphI, ShoI, NotI, BstXI, EcoRI, BamH1, SacI, KpnI, and HindIII cloning site, N-terminal peptide purified with ProBond resin and cleaved with enterokinase; Invitrogen), to mention just two, can be employed according to the invention.

Once a particular recombinant DNA molecule is identified and isolated, several methods known in the art may be used to propagate it. Once a suitable host system and growth conditions are established, recombinant expression vectors can be propagated and prepared in quantity. As previously explained, the expression vectors which can be used include, but are not limited to the following vectors or their derivatives: human or animal viruses such as vaccinia virus or adenovirus; insect viruses such as baculovirus; yeast vectors; bacteriophage vectors (e.g., lambda), and plasmid and cosmid DNA vectors, to name but a few.

Examples of unicellular hosts contemplated by the present invention include, but are not limited to E. coli Pseudonomas, Bacillus, Streptomyces, yeast, CHO, R1.1, B-W, L-M, COS1, COS7, BSC1, BSC40, BMT10 and Sf9 cells. In addition, a host cell strain may be chosen which modulates the expression of a variant allele comprising a human orphanin FQ/nociceptin receptor gene, or an isolated nucleic acid selectively hybridizable thereto, such that the gene product is modified and processed in the specific fashion desired. Different host cells have characteristic and specific mechanisms for the translational and post-translational processing and modification (e.g., glycosylation, cleavage [e.g., of signal sequence]) of proteins. Appropriate cell lines or host systems can be chosen to ensure the desired modification and processing of the foreign protein expressed. For example, expression in a bacterial system can be used to produce an nonglycosylated core protein product. However, a translocation signal sequence of an isolated variant allele of a human orphanin FQ/nociceptin receptor gene of the present invention, or an isolated nucleic acid selectively hybridizable thereto, expressed in bacteria may not be properly spliced. Expression in yeast can produce a glycosylated product. Expression in eukaryotic cells can increase the likelihood of “native” glycosylation and folding. Moreover, expression in mammalian cells can provide a tool for reconstituting, or constituting activity of the variant human orphanin FQ/nociceptin receptor gene. Furthermore, different vector/host expression systems may affect processing reactions, such as proteolytic cleavages, to a different extent.

Vectors are introduced into the desired unicellular hosts by methods known in the art, e.g., transfection, electroporation, microinjection, transduction, cell fusion, DEAE dextran, calcium phosphate precipitation, lipofection (lysosome fusion), use of a gene gun, or a DNA vector transporter (see, e.g., Wu et al., 1992, J. Biol. Chem. 267:963-967; Wu and Wu, 1988, J. Biol.

Chem. 263:14621-14624; Hartmut et al., Canadian Patent Application No. 2,012,311, filed Mar. 15, 1990).

Consequently, the present invention extends to a method for determining a susceptibility of a subject to a disease comprising removing a bodily sample comprising a first and second allele of a human orphanin FQ/nociceptin receptor gene from the subject, and determining whether either the first or second alleles, or both alleles comprise a DNA sequence having at least one variation in SEQ ID NO:1, wherein the variation comprises G-46A, GIVS I 135C, GIVS I 250A, GIVS I 251A, C510T, CIVS III 67T, A804G, C1026T, or C1126G.

Variant alleles of a human orphanin FQ/nociceptin receptor gene indicating increased or decrease susceptibility to diseases in the subject as described above, can be detected from cellular sources, such as, but not limited to, whole blood, epithelial cells obtained from the mouth, brain tissue biopsies, adipocytes, testes, heart, and the like. For example, cells can be obtained from an individual by biopsy and lysed, e.g., by freeze-thaw cycling, or treatment with a mild cytolytic detergent such as, but not limited to, TRITON X-100®, digitonin, NONIDET P (NP)-40®, saponin, and the like, or combinations thereof (see, e.g., International Patent 8 Publication WO 92/08981, published May 29, 1992). In yet another embodiment, samples containing both cells and body fluids can be used (see ibid.).

Other methods presently understood by a skilled artisan, and encompassed by the present invention, can also be used to detect the presence of either variation in either or both alleles of a human orphanin FQ/nociceptin receptor gene in a sample, and hence increased or decreased susceptibility to at least one disease of the subject relative to the susceptibility of at least one disease in a standard comprising alleles of the human orphanin FQ/nociceptin receptor gene comprising a DNA sequence of SEQ ID NO:1.

For example, an optionally detectably labeled isolated nucleic acid molecule selectively hybridizable to an isolated variant allele of a human orphanin FQ/nociceptin receptor gene comprising a DNA sequence having a variation in SEQ ID NO:1, wherein the variation comprises G-46A, GIVS I 135C, GIVS I 250A, GIVS I 251A, C510T, CIVS III 67T, A804G, C1026T, or C1126G, can be used in standard Northern hybridization analysis to detect the presence, and in some instances quantitate the level of transcription of such a variant allele of the present invention.

Alternatively, oligonucleotides of the invention can be used as PCR primers to amplify an allele of a human orphanin FQ/nociceptin receptor gene of the biological sample e.g., by reverse transcriptase-PCR, or amplification of the allele itself. The amplified mRNA or DNA can then be quantified or sequenced in order to determine the presence of a variant allele, and the susceptibility of the subject to addictive diseases. Furthermore, variations in SEQ ID NO:1, as described above, can be found by creation or deletion of restriction fragment length polymorphisms (RFLPs) not found in the predominant or “most common” allele, hybridization with a specific probe engineered to selectively hybridize to variation described, (or lack of hybridization with a probe specific for the predominant or “most common” allele), as well as by other techniques.

Furthermore, biochemical or immunochemical/biochemical (e.g., immunoprecipitation) techniques can be used to detect the presence and or level of expression of a variant allele of a human orphanin FQ/nociceptin receptor gene comprising a DNA sequence having a variation in SEQ ID NO:1 as described herein.

### Determining Susceptibility to Pain in a Subject

In yet another embodiment, the present invention extends to a method for determining a susceptibility to pain in a subject.

Hence, disclosed herein is a method of determining susceptibility of pain in a subject, comprising the steps of removing a bodily sample comprising a first and second allele of a human orphanin FQ/nociceptin receptor gene from the subject, and determining whether either the first or second alleles, or both alleles, comprise a DNA sequence having at least one variation in SEQ ID NO:1, wherein the variation comprises G-46A, GIVS I 135C, GIVS I 250A, GIVS I 251A, C510T, CIVS III 67T, A804G, C1026T, or C1126G.

The presence of at least one variation in either or both alleles of the human orphanin FQ/nociceptin receptor gene is expected to be indicative of the subject's increased or decreased susceptibility to pain relative to a person homozygous with respect to the predominant or “most common” allele comprising a human orphanin FQ/nociceptin receptor gene comprising a DNA sequence of SEQ ID NO:1.

Numerous methods presently available, and understood by the skilled artisan, can be used to “genotype” a subject in regards to the presence of a variant allele of a human orphanin FQ/nociceptin receptor gene in the genome of the subject. In particular, methods described above to ascertain increased or decreased susceptibility to addictive diseases have relevance in this embodiment of the present invention, and can readily be used herein. For example, Northern blot hybridization an isolated nucleic acid of the present invention selectively hybridizable to an isolated variant allele of a human orphanin FQ/nociceptin receptor gene comprising a DNA sequence having a variation of SEQ ID NO:1, wherein the variation comprises G-46A, GIVS I 135C, GIVS I 250A, GIVS I 251A, C510T, CIVS III 67T, A804G, C1026T, or C1126G, as a probe, along with RT-PCR, PCR, and numerous immunoassays described above, have applications herein.

Moreover, once susceptibility to pain in a subject has been determined, it is possible for attending medical professionals treating the subject for pain to administer an appropriate amount of pain reliever to the subject in order to induce analgesia. More specifically, an inappropriate amount of pain reliever is administered to a subject when either the subject is not relieved of pain, or the subject is exposed to potential deleterious side effects of the pain reliever, such as induction of addiction to the pain reliever, brain damage, or death.

However, since the amount of pain reliever administered to a subject is presently based principally on weight, information regarding the genotype of the subject with respect to the human orphanin FQ/nociceptin receptor gene can help increase accuracy in determining a therapeutically effective amount of pain reliever to administer in order to induce analgesia, making the use of pain relievers much safer for the subject.

Similarly, once ascertained, a susceptibility to addiction and response to human orphanin FQ/nociceptin receptor directed therapeutic agents, appropriate medications and dosages thereof can be determined for treatment of addictive diseases.

### Commercial Kits

Furthermore, as explained above, the present invention extends to commercial kits having applications in screening a bodily sample comprising DNA or RNA taken from a subject for the presence of a variant allele comprising a human orphanin FQ/nociceptin receptor comprising a DNA sequence having a variation in SEQ ID NO:1, wherein the variation comprises G-46A, GIVS I 135C, GIVS I 250A, GIVS I 251A, C510T, CIVS III 67T, A804G, C1026T, C1126G, or combinations thereof.

With information obtained from the use of a test kit of the present invention, an attending health profession can determine whether the subject has an susceptibility to pain relative to a standard, an increased susceptibility to at least one addictive disease relative to the susceptibility of a standard, a therapeutically effective amount of pain reliever to administer to the subject suffering from pain in order to induce analgesia in the subject relative to the therapeutically effective amount of pain reliever to administer to a standard in order to induce analgesia in the standard, or a therapeutically effective amount therapeutic agent to administer to a subject suffering from at least one addictive disease, relative to the therapeutically effective amount of therapeutic agent to administer to standard suffering from at least one addictive disease. Furthermore, such information can also be used to diagnose a disease or disorder related to a physiological function of the endogenous opioid system, nociception, neurotransmitter release (including dopamine, GABA, noradrenaline, and serotonin), anxiety and stress, learning, memory and cognition, alcohol self-administration, behavioral sensitization to cocaine, drug addition, opiate withdrawal and tolerance, food intake, immune function, cardiovascular function, renal function, gastrointestinal function, and motor function. In each use described above, the standard comprises a first and or second allele of a human orphanin FQ/nociceptin receptor gene comprising a DNA sequence of SEQ ID NO:1.

Accordingly, a test kit of the present invention for determining whether a subject comprises a variant allele of a human orphanin FQ/nociceptin receptor gene comprising a DNA sequence having a variation in SEQ ID NO:1, comprises means for detecting the presence of a variation in a first and or second allele comprising a human orphanin FQ/nociceptin receptor in a biological sample from a subject, and optimally packaged with directions for use of the kit. In one particular aspect, a test kit comprises an oligonucleotide probe(s) for binding to a variant allele of a human orphanin FQ/nociceptin receptor gene comprising a DNA sequence having a variation in SEQ ID NO:1; and means for detecting the level of binding of the probe to the variant allele, wherein detection binding of the probe to the variant allele indicates the presence of a variant comprising a human orphanin FQ/nociceptin receptor gene comprising a DNA sequence having a variation in SEQ ID NO:1, wherein the variation comprises G-46A, GIVS I 135C, GIVS I 250A, GIVS I 251A, C510T, CIVS III 67T, A804G, C1026T, C1126G, or combinations thereof.

The sequence of the oligonucleotide probe used in a commercial kit will determine which if any variation is present in an allele comprising a human orphanin FQ/nociceptin receptor gene.

Should no binding be detected, it is probable that no such variation exists in either allele of the subject.

More specifically, a commercial test kit of the present invention comprises:

a) PCR oligonucleotide primers suitable for detection of a variant allele of a human orphanin FQ/nociceptin receptor gene comprising a DNA sequence having a variation in SEQ ID NO:1, as set forth above,

b) other reagents; and

c) directions for use of the kit.

Examples of PCR oligonucleotide primer suitable for detection of an allele comprising a human orphanin FQ/nociceptin receptor gene comprising a DNA sequence having a variation in SEQ ID NO:1 can be readily produced by a person of ordinary skill in the art with teaching set forth herein, and variations of SEQ ID NO:1 also set forth herein.

The present invention may be better understood by reference to the following non-limiting Example, which is provided as exemplary of the invention. The following Example is presented in order to more fully illustrate the preferred embodiments of the invention. It should in no way be construed, however, as limiting the broad scope of the invention.

### EXAMPLE

To identify polymorphisms of the human orphanin FQ/nociceptin receptor, a PCR-based strategy was used to amplify the coding regions of the orphanin FQ/nociceptin receptor gene, and to determine the DNA sequence of the amplified exons. Using this method DNA samples were sequenced from 129 unrelated subjects.

Study subjects and procedures. Addictive disease patients, specifically long-term heroin addicts currently in chronic methadone maintenance treatment, and normal control subjects with no history of any drug or alcohol abuse, and individuals with non-opiate drug abuse and dependence were extensively characterized with respect to drug abuse, the addictive diseases, psychological and psychiatric profiles, and medical and ethnic family backgrounds. Unrelated study subjects who were former heroin addicts were referred from methadone treatment clinics in the greater New York City area, primarily those associated with The Biology of Addictive Diseases Laboratory located at The Rockefeller University. These clinics are the Adolescent Development Program and Adult Clinic at the New York Hospital-Cornell Medical Center. Previously heroin-addicted patients admitted to the study conformed to the federally regulated criteria for admission to a methadone maintenance program, that is, one or more years of daily multiple-dose self-administration of heroin or other opiates with the development of tolerance, dependence, and drug-seeking behavior. Current or prior abuse of other drugs was not used as an exclusion criterion for this group as long as opioid abuse continued to be the primary diagnosis.

Unrelated healthy volunteer subjects were recruited primarily through posting of notices and newspaper advertisements or referral by physicians or staff at the Rockefeller University Hospital. Individuals with continuing drug or alcohol abuse or prior extended periods of regular abuse were also studied.

Both addictive disease patients and normal volunteers admitted to the study were assessed by a psychiatrist or research nurse with several psychiatric and psychological instruments as well as the Addiction Severity Index. Study subjects were also administered a detailed personal and medical and special addictive disease questionnaire as well as a family history medical and addictive disease questionnaire designed to provide information regarding substance abuse and major mental illness of first and second degree relatives. Study subjects provided detailed information regarding family origin and ethnic background, including country or geographic area of birth. This information was obtained for both the study subjects themselves and their immediate ancestors (parents, grandparents and great-grandparents), to the extent that the information was known by the study subjects. Study subjects were classified into five groups: African-American, Caucasian, Hispanic (Caribbean and Central or South American origin), Native North American, and Other. The detailed ancestral information collected by the family origin questionnaire allowed classification of study subjects into defined categories. Following psychiatric and behavioral assessment and informed consent and family history acquisition, venipuncture on the study subject was performed, and a blood specimen was taken. Blood samples were processed for DNA extraction and EBV transformation to create stable cell lines that were stored for future studies. All blood samples were coded; the psychiatrists and nurses who performed psychiatric and psychological assessments were blind to the genotypes of the study subjects, and the identity and categorization of the study subjects was unknown to the laboratory research personnel.

By sequencing PCR-amplified DNA from the study subjects, it was determined that the previously reported sequence for the human orphanin FQ/nociceptin receptor was the most common allele found in the study population. Nine new silent polymorphisms were also identified: G-46A (G minus 46 A) (SEQ ID No:3), located in the 5′ untranslated region; GIVS I 135C (SEQ ID No:4), located in intron I; GIVS I 250A (SEQ ID No:5), located in intron I; GIVS I 251A (SEQ ID No:6), located in intron I; C510T (SEQ ID No:7), a silent mutation located in the coding region; CIVS III 67T (SEQ ID No:8), located in intron III; A804G (SEQ ID No:9), a silent mutation located in the coding region; C1026T (SEQ ID No:10), a silent mutation located in the coding region; and C1126G (SEQ ID No:11), located in the 3′ untranslated region. For the purpose of this study, the term “most common” was used to denote the predominant orphanin FQ/nociceptin receptor allele (SEQ. ID NO:1) and the corresponding receptor that was originally reported by cDNA cloning and the term “variant” to denote the allelic genes/receptors containing polymorphic variations.

Moreover, during the course of the studies herein, a new 511-nucleotide intron was discovered located between bases −34 and −33 of the mRNA. It is designated herein “intervening sequence I (IVS I)”. The inventors note the existence of a previously-reported 118-nucleotide intron between base +589 and base +590 (see Mollereau et al., 1994), which herein is referred to as “intervening sequence III (IVS III)”. The numbering system used herein is based on the prototypic mRNA sequence as reported in Molleareau and colleagues (1994) and not on splice variant forms subsequently identified (Wick et al., 1995; Peluso et al., 1998). SNPs which fall in the intron sequences are designated by the intron number (IVS I or IVS III) followed by the number of bases from the first base of that intron sequence.

The polymorphisms and number of individuals in which they were identified are as follows:

| | | | Allele frequency of |
|---|---|---|---|
| Variant | Position | # of individuals | variant SNP |
| G-46A | 5′ untranslated region | 7 heterozygous G/A | 0.031 |
| GIVS I 135C | Intron I | 1 heterozygous G/C | 0.004 |
| GIVS I 250A | Intron I | 10 heterozygous G/A | 0.044 |
| GIVS I 251A | Intron I | 1 heterozygous G/A | 0.004 |
| C510T | Coding region | 23 heterozygous C/T, | 0.136 |
| | | 4 homozygous T/T | |
| CIVS III 67T | Intron III | 28 heterozygous C/T, | 0.175 |
| | | 6 homozygous T/T | |
| A804G | Coding region | 4 heterozygous A/G | 0.018 |
| C1026T | Coding region | 2 heterozygous C/T | 0.009 |
| C1126G | 3′ untranslated region | 1 heterozygous C/G | 0.004 |

In addition, a number of double heterozygotes were identified. The following numbers of individuals heterozygous for two SNPs in the hORL1 gene were identified in a cohort of 114 subjects.

| Heterozygote SNPS | Number of individuals |
|---|---|
| G-46A and CIVS III 67T | 3 |
| GIVS I 135C and C510T | 1 |
| CIVS I 250A and C510T | 2 |
| CIVS I 250A and CIVS III 67T | 4 |
| CIVS I 251A and CIVS III 67T | 1 |
| C510T and CIVS III 67T | 3 |
| CIVS III 67T and C1026T | 2 |

The present invention is not to be limited in scope by the specific embodiments describe herein. Indeed, various modifications of the invention in addition to those described herein will become apparent to those skilled in the art from the foregoing description and the accompanying figures. Such modifications are intended to fall within the scope of the appended claims.

Various publications are cited herein, the disclosures of which are incorporated by reference in their entireties.

Bunzow JR, Saez C, Mortrud M, Bouvier C, Williams J T, Low M, Grandy D K. 1994 Molecular cloning and tissue distribution of a putative member of the rat opioid receptor gene family that is not a mu, delta or kappa opioid receptor type FEBS Lett. 347, 284-288.

Fukuda K, Kato S, Mori K, Nishi M. Takeshima H, Iwabe N, Miyata T, Houtani T Sugimoto T. 1994 cDNA cloning and regional distribution of a novel member of the opioid receptor family FEBS Lett. 343, 42-46.

Mollereau C, Parmentier M, Mailleux P, Butour J L, Moisand C, Chalon P, Caput D, Vassart G, Meunier J C. 1994. ORL1, a novel member of the opioid receptor family: Cloning functional expression and localization. FEBS Lett. 341, 33-38.

Wang J B, Johnson P S, Imai Y, Persico A M, Ozenberger B A, Eppler C M, Uhl G R. 1994 cDNA cloning of an orphan opiate receptor gene family member and its splice variant FEBS Lett. 348, 75-79.

Wick M J, Minnerath S R, Roy S, Ramakrishnan S, Loh H H. 1995 Expression of alternate forms of brain opioid ‘orphan’ receptor mRNA in activated human peripheral blood lymphocytes and lymphocytic cell lines. Mol Brain Res. 32, 342-347.

Peluso J, LaForge K S, Matthes H W, Kreek M J, Kieffer B L, Gavériaux-Ruff C. 1998. Distribution of nociceptin/orphanin FQ receptor transcript in human central nervous system and immune cells. J. Neuroimmunol. 81, 184-192.

What is claimed is:

1. An isolated variant allele of a human orphanin FQ/nociceptin receptor gene, comprising a DNA sequence having at least one variation in SEQ ID NO:1, wherein said variation comprises G-46A, GIVS I 135C, GIVS I 250A, GIVS I 251A, C510T, CIVS III 67T, A804G, C1026T, C1126G, or any combination thereof.

2. The isolated variant allele of claim 1, detectably labeled.

3. The isolated variant allele of claim 2, wherein said detectable label comprises a radioactive element, a chemical which fluoresces, or an enzyme.

4. An isolated nucleic acid molecule selectively hybridizable to the isolated variant allele of claim 1.

5. The isolated nucleic acid molecule of claim 4, detectably labeled.

6. The isolated nucleic acid molecule of claim 5, wherein said detectable label comprises a radioactive element, a chemical that fluoresces, or an enzyme.

7. A cloning vector comprising an isolated variant allele of a human orphanin FQ/nociceptin receptor gene and an origin of replication, wherein said variant allele comprises a DNA sequence having at least one variation in SEQ ID NO:1, wherein said variation comprises G-46A, GIVS I 135C, GIVS I 250A, GIVS I 251A, C510T, CIVS III 67T, A804G, C1026T, C1126G, or any combination thereof.

8. A cloning vector comprising an origin of replication and an isolated nucleic acid molecule selectively hybridizable to an isolated variant allele of a human orphanin FQ/nociceptin receptor gene, wherein said variant allele comprises a DNA sequence having at least one variation in SEQ ID NO:1, wherein said at least one variation comprises G-46A, GIVS I 135C, GIVS I 250A, GIVS I 251A, C510T, CIVS III 67T, A804G, C1026T, C1126G, or any combination thereof.

9. The cloning vector of either of claim 7 or 8, wherein said cloning vector comprises of E. coli, bacteriophages, plasmids, or pUC plasmid derivatives.

10. The cloning vector of claim 9, wherein bacteriophages further comprise lambda derivatives, plasmids further comprise pBR322 derivatives, and pUC plasmid derivatives further comprise pGEX vectors, or pmal-c, pFLAG.

11. An expression vector comprising an isolated variant allele of a human orphanin FQ/nociceptin receptor gene comprising a DNA sequence having a variation in SEQ ID NO:1, wherein said variation comprises G-46A, GIVS I 135C, GIVS I 250A, GIVS I 251A, C510T, CIVS III 67T, A804G, C1026T, C1126G, or any combination thereof.

12. An expression vector comprising an isolated nucleic acid molecule selectively hybridizable to an isolated variant allele of a human orphanin FQ/nociceptin receptor gene, wherein said isolated nucleic acid molecule is operatively associated with a promoter, and said variant allele comprises a DNA sequence having at least one variation in SEQ ID NO:1, wherein said at least one variation comprises G-46A, GIVS I 135C, GIVS I 250A, GIVS I 251A, C510T, CIVS III 67T, A804G, C1026T, C1126G, or any combination thereof.

13. The expression vector of either of claim 11 or 12, wherein said promoter comprises immediate early promoters of hCMV, early promoters of SV40, early promoters of adenovirus, early promoters of vaccinia, early promoters of polyoma, late promoters of SV40, late promoters of adenovirus, late promoters of vaccinia, late promoters of polyoma, the lac the trp system, the TAC system, the TRC system, the major operator and promoter regions of phage lambda, control regions of fd coat protein, 3-phosphoglycerate kinase promoter, acid phosphatase promoter, or promoters of yeast a mating factor.

14. A unicellular host transformed or transfected with an expression vector comprising an isolated variant allele of a human orphanin FQ/nociceptin receptor gene operatively associated with a promoter, wherein said variant allele comprises a DNA sequence having at least one variation in SEQ ID NO:1, wherein said at least one variation comprises G-46A, GIVS I 135C, GIVS I 250A, GIVS I 251A, C510T, CIVS III 67T, A804G, C1026T, C1126G, or any combination thereof.

15. A unicellular host transformed with an expression vector comprising an isolated nucleic acid molecule selectively hybridizable to an isolated variant allele of a human orphanin FQ/nociceptin receptor gene, wherein said isolated nucleic acid molecule is operatively associated with a promoter, and said variant allele comprises a DNA sequence having at least one variation in SEQ ID NO:1, wherein said at least one variation comprises G-46A, GIVS I 135C, GIVS I 250A, GIVS I 251A, C510T, CIVS III 67T, A804G, C1026T, C1126G, or any combination thereof.

16. The unicellular host of either of claim 14 or 15, wherein said host comprises E. coli, Pseudomonas, Bacillus, Streptomyces, yeast, CHO, R1.1, B-W, L-M, COS1, COS7, BSC1, BSC40, BMT10 or Sf9 cells.

17. A method for determining a susceptibility in a subject to at least one physiological response, condition or disease related to the endogenous opioid system, nociception, neurotransmitter release (including dopamine, GABA, noradrenaline, and serotonin), anxiety and stre7ss, learning, memory and cognition, alcohol self-administration, behavioral sensitization to cocaine, drug addiction, opiate withdrawal and tolerance, food intake, immune function, cardiovascular function, renal function, gastrointestinal function, or motor function, comprising the steps of:
a) removing a bodily sample from said subject, wherein said sample comprises a first and second allele comprising a human orphanin FQ/nociceptin receptor gene;
b) determining whether said human orphanin FQ/nociceptin receptor gene of said first allele comprises a DNA sequence having at least one variation in SEQ ID NO:1, wherein said variation comprises G-46A, GIVS I 135C, GIVS I 250A, GIVS I 251A, C510T, CIVS III 67T, A804G, C1026T, C1126G, or any combination thereof, such that the presence of said at least one variation in said human orphanin FQ/nociceptin receptor gene of said first allele is expected to be indicative of the subject's susceptibility to at least one said physiological response, condition or disease relative to the susceptibility to said at least one said physiological response, condition or disease in a standard.

18. The method for determining a susceptibility to at least one addictive disease of claim 17, further comprising the step of determining whether said human orphanin FQ/nociceptin receptor gene of said second allele comprises a DNA sequence having at least one variation in SEQ ID NO:1, wherein said variation comprises G-46A, GIVS I 135C, GIVS I 250A, GIVS I 251A, C510T, CIVS III 67T, A804G, C1026T, C1126G, or any combination thereof, such that the presence of said at least one variation in said human orphanin FQ/nociceptin receptor gene of said second allele is expected to be indicative of the subject's susceptibility to said at least one physiological response, condition or disease relative to the susceptibility to said at least one physiological response, condition or disease in said standard.

19. The method of either of claim 19 wherein said at least one addictive disease comprises opioid addiction; cocaine addiction or addiction to other psychostimulants; nicotine addiction; barbituate or sedative hypnotic addiction; anxiolytic addiction; or alcohol addiction.

20. A method for determining a susceptibility to pain in a subject relative to a susceptibility of pain in a standard, wherein the method comprises the steps of:
a) removing a bodily sample from said subject, wherein said sample comprises a first and second allele comprising a human orphanin FQ/nociceptin receptor gene;
b) determining whether said human orphanin FQ/nociceptin receptor gene of said first allele comprises a DNA sequence having at least one variation in SEQ ID NO:1, wherein said variation comprises G-46A, GIVS I 135C, GIVS I 250A, GIVS I 251A, C510T, CIVS III 67T, A804G, C1026T, C1126G, or any combination thereof, such that the presence of said at least one variation in said human orphanin FQ/nociceptin receptor gene of said first allele is expected to be indicative of susceptibility to pain in said subject relative to susceptibility to pain in said standard, wherein said first allele of said standard comprises a human orphanin FQ/nociceptin receptor gene comprising a DNA sequence of SEQ ID NO:1.

21. The method of claim 20 for determining a susceptibility to pain in a subject, further comprising the step of determining whether said second allele of said bodily sample comprises a human orphanin FQ/nociceptin receptor gene comprising a DNA sequence having at least one variation in SEQ ID NO:1, wherein said variation comprises G-46A, GIVS I 135C, GIVS I 250A, GIVS I 251A, C510T, CIVS III 67T, A804G, C1026T, C1126G, or any combination thereof, such that the presence of said at least one variation in said second allele is expected to be indicative of susceptibility to pain in said subject relative to susceptibility of pain in said standard, wherein said second allele of said standard comprises a human orphanin FQ/nociceptin receptor gene comprising a DNA sequence of SEQ ID NO:1.

22. A method for determining a therapeutically effective amount of pain reliever to administer to a subject in order to induce analgesia in said subject relative to a therapeutically effective amount of pain reliever to administer to a standard in order to induce analgesia in said standard, wherein the method comprises determining a susceptibility to pain in said subject relative to susceptibility to pain in said standard, wherein susceptibility to pain in said subject is expected to be indicative of said therapeutically effective amount of pain reliever to administer to said subject to induce analgesia in said subject relative to said therapeutically effective amount of pain reliever to administer to said standard to induce analgesia in said standard.

23. The method of claim 22 for determining a therapeutically effective amount of pain reliever to administer to said subject, wherein determining susceptibility to pain in said subject comprises the steps of:
a) removing a bodily sample from said subject, wherein said sample comprises a first and second allele comprising a human orphanin FQ/nociceptin receptor gene; and
b) determining whether said first allele comprises a human orphanin FQ/nociceptin receptor gene comprising a DNA sequence having at least one variation in SEQ ID NO:1, wherein said at least one variation comprises G-46A, GIVS I 135C, GIVS I 250A, GIVS I 251A, C510T, CIVS II 67T, A804G, C1026T, C1126G, or any combination thereof, wherein the presence of said at least one variation in said human orphanin FQ/nociceptin receptor gene of said first allele is expected to be indicative of the subject's susceptibility to pain relative to said to susceptibility of pain in said standard, wherein said first allele of said standard comprises a human orphanin FQ/nociceptin receptor gene comprising a DNA sequence of SEQ ID NO:1, such that said therapeutically effective amount of pain reliever to administer to the subject in order to induce analgesia is related to said susceptibility to pain in said subject relative to susceptibility to pain in said standard.

24. The method of claim 23, wherein determining susceptibility to pain in said subject relative to susceptibility to pain in said standard further comprises the step of determining whether said second allele of said bodily sample from said subject comprises a human orphanin FQ/nociceptin receptor gene comprising a DNA sequence having at least one variation in SEQ ID NO:1, wherein said at least one variation comprises G-46A, GIVS I 135C, GIVS I 250A, GIVS I 251A, C510T, CIVS III 67T, A804G, C 1026T, C1126G, or any combination thereof, such that the presence of said at least one variation in said second allele is expected to be indicative of susceptibility to pain in said subject relative to susceptibility to pain in said standard, wherein said second allele of said standard comprises a human orphanin FQ/nociceptin receptor gene comprising a DNA sequence of SEQ ID NO:1, and the therapeutically effective amount of pain reliever to administer to said subject to induce analgesia in said subject is related to the presence of said at least one variation in said human orphanin FQ/nociceptin receptor gene of said second allele of said bodily sample from said subject.

25. A method for determining a therapeutically effective amount of therapeutic agent to administer to a subject suffering from at least one addictive disease to treat the at least one addictive disease in said subject relative to a therapeutically effective amount of therapeutic agent to administer to a standard suffering from the at least one addictive disease to treat the at least one addictive disease in said standard, wherein the method comprises the steps of:
a) removing a bodily sample from said subject, wherein said sample comprises a first and second allele comprising a human orphanin FQ/nociceptin receptor gene; and
b) determining whether said first allele comprises a human orphanin FQ/nociceptin receptor gene comprising a DNA sequence having at least one variation in SEQ ID NO:1, wherein said variation comprises G-46A, GIVS I 135C, GIVS I 250A, GIVS I 251A, C510T, CIVS III 67T, A804G, C1026T, C1126G, or any combination thereof,
wherein the presence of said at least one variation in said human orphanin FQ/nociceptin receptor gene of said first allele is expected to be indicative of the therapeutically effective amount of said therapeutic agent to administer to the subject to treat said at least one addictive disease in said subject relative to said therapeutically effective amount of said therapeutic agent to administer to said standard to treat said at least one addictive disease in said standard, wherein said first allele of said standard comprises a human orphanin FQ/nociceptin receptor gene comprising a DNA sequence of SEQ ID NO:1.

26. The method of claim 25 for determining a therapeutically effective amount of therapeutic agent to administer to a subject suffering from said at least one addictive disease to treat said at least one addictive disease, relative to said therapeutically effective amount of said therapeutic agent administered to said standard suffering from said at least one addictive disease to treat said at least one addictive disease in said standard, further comprising the step of determining whether said second allele of said bodily sample from said subject comprises a human orphanin FQ/nociceptin receptor gene comprising a DNA sequence having at least one variation in SEQ ID NO:1, wherein said variation comprises G-46A, GIVS I 135C, GIVS I 250A, GIVS I 251A, C510T, CIVS III 67T, A804G, C1026T, C1126G, or any combination thereof, such that the presence of said at least one variation in said second allele related to said therapeutically effective amount of said therapeutic agent administered to said subject to treat said at least one addictive disease in said subject relative to said therapeutically effective amount of said therapeutic agent to administer to said standard to treat said at least one addictive disease in said standard, wherein said second allele of said standard comprises a human orphanin FQ/nociceptin receptor gene comprising a DNA sequence of SEQIDNO:1.

27. The method of either of claim 25 or 26, wherein said at least one addictive disease comprises opioid addiction; cocaine addiction or addiction to other psychostimulants; nicotine addiction; barbiturate or sedative hypnotic addiction; anxiolytic addiction; or alcohol addiction.

28. A commercial test kit may for determining the presence of at least one variation in a human orphanin FQ/nociceptin receptor gene of an allele in a bodily sample taken from a subject, wherein the commercial test kit comprises:
a) PCR oligonucleotide primers suitable for detection of an allele comprising a human orphanin FQ/nociceptin receptor gene comprising a DNA sequence having at least one variation in SEQ ID NO:1 comprising G-46A, GIVS I 135C, GIVS I 250A, GIVS I 251A, C510T, CIVS III 67T, A804G, C1026T, C1126G, or any combination thereof;
b) other reagents; and
c) directions for use of the kit.

29. A nucleic acid comprising an intron of the human orphanin FQ/nociceptin receptor gene as set forth in SEQ ID No:2.

30. A nucleic acid as set forth in SEQ ID No:2.
